SEQUENCE LISTING (27 sequences)

SEQ ID NO: 1 - DNA, 24 bp, Artificial sequence
  Synthetic oligonucleotide
tcgtcgttttgtcgttttgtcgtt

SEQ ID NO: 2 - DNA, 34 bp, Artificial sequence
  Synthetic oligonucleotide
tcnnnnnnnnnnnnnnnnnnnnnnnnntnncgnn

SEQ ID NO: 3 - DNA, 12 bp, Artificial sequence
  Synthetic oligonucleotide
cgacgttcgtcg

SEQ ID NO: 4 - DNA, 13 bp, Artificial sequence
  Synthetic oligonucleotide
cggcgccgtgccg

SEQ ID NO: 5 - DNA, 12 bp, Artificial sequence
  Synthetic oligonucleotide
ccccccgggggg

SEQ ID NO: 6 - DNA, 12 bp, Artificial sequence
  Synthetic oligonucleotide
ggggggcccccc

SEQ ID NO: 7 - DNA, 10 bp, Artificial sequence
  Synthetic oligonucleotide
cccccggggg

SEQ ID NO: 8 - DNA, 10 bp, Artificial sequence
  Synthetic oligonucleotide
gggggccccc

SEQ ID NO: 9 - DNA, 22 bp, Artificial sequence
  Synthetic oligonucleotide
tcgcgtcgttcggcgcgcgccg

SEQ ID NO: 10 - DNA, 23 bp, Artificial sequence
  Synthetic oligonucleotide
tcgtcgacgttcggcgcgcgccg

SEQ ID NO: 11 - DNA, 21 bp, Artificial sequence
  Synthetic oligonucleotide
tcggacgttcggcgcgcgccg

SEQ ID NO: 12 - DNA, 19 bp, Artificial sequence
  Synthetic oligonucleotide
tcggacgttcggcgcgccg

SEQ ID NO: 13 - DNA, 20 bp, Artificial sequence
  Synthetic oligonucleotide
tcgcgtcgttcggcgcgccg

SEQ ID NO: 14 - DNA, 20 bp, Artificial sequence
  Synthetic oligonucleotide
tcgacgttcggcgcgcgccg

SEQ ID NO: 15 - DNA, 18 bp, Artificial sequence
  Synthetic oligonucleotide
tcgacgttcggcgcgccg

SEQ ID NO: 16 - DNA, 18 bp, Artificial sequence
  Synthetic oligonucleotide
tcgcgtcgttcggcgccg

SEQ ID NO: 17 - DNA, 22 bp, Artificial sequence
  Synthetic oligonucleotide
tcgcgacgttcggcgcgcgccg

SEQ ID NO: 18 - DNA, 22 bp, Artificial sequence
  Synthetic oligonucleotide
tcggcgcgcgccgtgctgcttt

SEQ ID NO: 19 - DNA, 24 bp, Artificial sequence
  Synthetic oligonucleotide
tngtngttttgtngttttgtngtt

SEQ ID NO: 20 - DNA, 24 bp, Artificial sequence
  Synthetic oligonucleotide
tgctgcttttgtgcttttgtgctt

SEQ ID NO: 21 - DNA, 24 bp, Artificial sequence
  Synthetic oligonucleotide
tgctgcttttgtgcttttgtgctt

SEQ ID NO: 22 - DNA, 24 bp, Artificial sequence
  Synthetic oligonucleotide
gtgctcctttgttgttctgtgttt

SEQ ID NO: 23 - DNA, 24 bp, Artificial sequence
  Synthetic oligonucleotide
aagcacaaaagcacaaaagcagca

SEQ ID NO: 24 - DNA, 22 bp, Artificial sequence
  Synthetic oligonucleotide
tgctggcctcctggcctggtgc

SEQ ID NO: 25 - DNA, 20 bp, Artificial sequence
  Synthetic oligonucleotide
tgtgcttttttttttttttt

SEQ ID NO: 26 - DNA, 20 bp, Artificial sequence
  Synthetic oligonucleotide
tccaggacttctctcaggtt

SEQ ID NO: 27 - DNA, 20 bp, Artificial sequence
  Synthetic oligonucleotide
gccaggacacctcacaggat

(19) United States
(12) Patent Application Publication
(10) Pub. No.: US 2006/0019923 A1
(43) Pub. Date: Jan. 26, 2006

(54) Methods and compositions for inducing innate immune responses

(75) Inventors: Heather L. Davis, Dunrobin (CA); Michael J. McCluskie, Ottawa (CA); Debra P. Drane, Bullengarook (AU)

Correspondence Address: WOLF GREENFIELD & SACKS, PC;FEDERAL RESERVE PLAZA, 600 ATLANTIC AVENUE, BOSTON, MA, 02210-2211, US

(73) Assignees: Coley Pharmaceutical Group, Ltd., Ottawa (Kanata) (CA); CSL Limited, Parkville (AU)

(21) Appl. No.: 11/184,065

(22) Filed: Jul. 18, 2005

Related U.S. Application Data
(60) Provisional application No. 60/589,258, filed on Jul. 18, 2004.

(51) Int. Cl. A61K 48/00

(52) U.S. Cl. 514/44

(57) ABSTRACT

The invention relates to TLR ligand formulations that comprise immune stimulating complexes and their use in inducing innate immunity.

### RELATED APPLICATION

This application claims priority to U.S. Provisional Application having Ser. No. 60/589,258 entitled “METHODS AND COMPOSITIONS FOR INDUCING INNATE IMMUNE RESPONSES” filed Jul. 18, 2004, the entire contents of which are incorporated by reference herein.

### FIELD OF THE INVENTION

The present invention relates generally to TLR ligand and immune stimulating complexes and their use in inducing innate immunity.

### BACKGROUND OF THE INVENTION

Bacterial DNA has immune stimulatory effects to activate B cells and natural killer cells, but vertebrate DNA does not (Tokunaga, T., et al., 1988. Jpn. J. Cancer Res. 79:682-686; Tokunaga, T., et al., 1984, JNCI 72:955-962; Messina, J. P., et al., 1991, J. Immunol. 147:1759-1764; and reviewed in Krieg, 1998, In: Applied Oligonucleotide Technology, C. A. Stein and A. M. Krieg, (Eds.), John Wiley and Sons, Inc., New York, N.Y., pp. 431-448). It is now understood that these immune stimulatory effects of bacterial DNA are a result of the presence of unmethylated CpG dinucleotides (i.e., an unmethylated cytosine attached to guanosine) in particular base contexts (CpG motifs), which are common in bacterial DNA, but comprise methylated cytosines and are underrepresented in vertebrate DNA (Krieg et al, 1995 Nature 374:546-549; Krieg, 1999 Biochim. Biophys. Acta 93321:1-10). The immune stimulatory effects of bacterial DNA can be mimicked with synthetic oligonucleotides (ODN) containing these CpG motifs. Such CpG ODN have highly stimulatory effects on human and murine leukocytes such as inducing B cell proliferation, cytokine and immunoglobulin secretion, natural killer (NK) cell lytic activity, and IFN-γ secretion; and activating dendritic cells (DCs) and other antigen presenting cells to express co-stimulatory molecules and secrete cytokines, especially the Th1-like cytokines that are important in promoting the development of Th1-like T cell responses. These immune stimulatory effects of native phosphodiester backbone CpG ODN are highly CpG specific in that the effects are dramatically reduced if the cytosine residue of the CpG motif is methylated, or if the CpG motif is changed to a GpC or otherwise eliminated or altered (Krieg et al, 1995 Nature 374:546-549; Hartmann et al, 1999 Proc. Natl. Acad. Sci USA 96:9305-10).

Animals have evolved to possess a variety of mechanisms to protect themselves against foreign substances such as microbes. These include physical barriers, phagocytic cells in the blood and tissues, natural killer cells and various blood-borne molecules. Some of these mechanisms are present prior to exposure to infectious microbes or foreign substances. Additionally, they do not discriminate between most foreign substances. And generally, they are not enhanced to any great extent by exposure to the foreign substance. As a result, these mechanisms are the host's first line of defense against invasion by foreign substances. Although limited in some sense, they are also the only line of defense until the adaptive or acquire immune response is triggered. The ability of a subject to mount an innate immune response may vary from subject to subject. These differences can control whether an infection is resolved without any or at least substantial symptoms, or whether the subject experiences an infection and its associated myriad of symptoms. Given its importance as a first line of defense, therapies which promote innate immunity are desirable. For example, a more robust innate immune response would overcome the need for more diverse antibiotics in this age of multi-resistant microbes.

### SUMMARY OF THE INVENTION

The invention is based in part on the unexpected finding that the inert TLR ligands can be transformed into immunostimulatory TLR ligands by combining and administering them with immune stimulating complexes. Inert TLR ligands are TLR ligands that prior to the invention have not been observed to be immunostimulatory, or which have at most been observed to be poorly immunostimulatory (i.e., at or around the immune stimulation level of a control molecule in previous assays in the absence of immune stimulating complexes). This observation suggests that the lack of immunostimulation observed with these TLR ligands (when used in the absence of an immune stimulating complex) may be due to their inefficient delivery to cells and receptors (e.g., the TLR family of receptors). Thus, the invention transforms a number of immunologically inert TLR ligands into immunostimulatory agents as a result of their formulation. Accordingly, inert TLR ligands when used together with immune stimulating complexes of the invention are useful in inducing innate immunity.

Thus, in one aspect, the invention provides a method for inducing an innate immune response comprising administering to a subject in need thereof an inert TLR ligand and an immune stimulating complex, in an amount effective to induce an innate immune response.

In one embodiment, the innate immune response comprises activation of natural killer (NK) cell activity. NK cells are part of the innate immune system and as such are involved in the first line of defense against pathogens. In another embodiment, the innate immune response comprises production and/or secretion of one or more cytokines or growth factors such as for example IFN-alpha, TNF-alpha, IL-1, IL-6, IL-10, IL-12 and IFN-gamma. Innate immunity may further comprise the involvement of macrophages, dendritic cells and monocytes.

In one embodiment, the inert TLR ligand is incorporated into the immune stimulating complex. In another embodiment, the inert TLR ligand is simply associated (e.g., non-covalently and non-ionically) with the complex.

As used herein, a formulation comprising an inert TLR ligand and an immune stimulatory complex is referred to as an inert TLR ligand/complex formulation.

In one embodiment, the formulation is made by mixing together the inert TLR ligand and the immune stimulating complex. In another embodiment, the inert TLR ligand intrinsically comprises or is extrinsically modified to comprise a moiety that is incorporated within the immune stimulating complex such as a sterol (e.g., cholesterol) or a saponin. The inert TLR ligand may also comprise (intrinsically or extrinsically) a lipidated tag such as but not limited to a palmitic tag, an oleic tag, etc. For example, the TLR ligand may be sterol-linked, glycoside-linked (e.g., saponin-linked), phospholipid-liked, and the like. The inert TLR ligand is then incorporated into the complex by virtue of the moiety that forms part of the complex.

The inert TLR ligand may be an oligonucleotide which in turn may comprise ribonucleotides or deoxyribonucleotides. In one embodiment, the oligonucleotide has a partially or wholly modified phosphate backbone, such as a backbone that is partially or wholly phosphorothioate. The TLR ligand may or may not comprise a palindrome.

Immune stimulating complexes are complexes that are comprised of at least a sterol and a saponin. They may optionally contain a phospholipid, or other lipid moiety, but this is not specifically required to observe the effects described herein. Examples of immune stimulating complexes include ISCOM® and ISCOMATRIX® adjuvants. The immune stimulating complex may be referred to herein as a sterol/saponin complex or formulation.

The inert TLR ligand may be present in a proportion of complexes (e.g., at least 25%, at least 40%, at least 50%, at least 75%, at least 80%, at least 90%, at least 95%, at least 99%, or all complexes contain the inert TLR ligand). In one embodiment, the inert TLR ligand and the immune stimulating complex are administered either intramuscularly or subcutaneously.

The method can be directed to various therapeutic or prophylactic settings including subjects having or at risk of having various conditions or diseases. In one embodiment, the subject has or is at risk of developing a cancer. Such a subject might also be at risk of developing an infectious disease and thus the method is a method for preventing or treating the cancer or an infectious disease (or both) in the subject. Opportunistic infectious diseases are common in immunocompromised subjects such as cancer patients undergoing anti-cancer treatment.

In one embodiment, the cancer is a carcinoma or a sarcoma. The cancer may be selected from the group consisting of biliary tract cancer, bone cancer, brain and CNS cancer, breast cancer, cervical cancer, choriocarcinoma, colon cancer, connective tissue cancer, endometrial cancer, esophageal cancer, eye cancer, gastric cancer, Hodgkin's lymphoma, intraepithelial neoplasm, larynx cancer, liver cancer, lung cancer (e.g. small cell and non-small cell cancer), lymphoma, melanoma, neuroblastoma, oral cancer, oral cavity cancer, ovarian cancer, pancreatic cancer, prostate cancer, rectal cancer, renal cancer, skin cancer, testicular cancer and thyroid cancer.

In another embodiment, the subject has or is at risk of developing an infection. The infection may be selected from the group consisting of a bacterial infection, a viral infection, a fungal infection, a parasitic infection and a mycobacterial infection. In one embodiment, the infection is a chronic viral infection such as but not limited to hepatitis B infection, hepatitis C infection, HIV infection, HSV infection or HPV infection. In some embodiments, the parasite infection is an intracellular parasite infection. In another embodiment, the parasite infection is a non-helminthic parasite infection. Other examples of each microbial infection are recited herein.

In another embodiment, the subject has or is at risk of developing a prion disease.

In another embodiment, the subject has or is at risk of developing an allergy or asthma.

The composition may be administered by any route, but in some embodiments, subcutaneous or intramuscular routes are preferred.

In one embodiment, the method further comprises administering a therapeutic regimen to the subject. The therapeutic regimen may be surgery, radiation or chemotherapy. Chemotherapy may be but is not limited to anti-cancer agents, anti-bacterial agents, anti-viral agents, anti-fungal agents, anti-parasite agents, anti-mycobacterial agents, anti-allergy agents and anti-asthma agents. The therapeutic regimen may also be antibody therapy. In embodiments directed towards treatment of subjects having or at risk of developing cancer, the method may further comprise administration of interferon-alpha, either within or separate from the TLR ligand and the immune stimulating complex.

In some embodiments the subject is a human, and in other embodiments the subject is a non-human vertebrate selected from the group consisting of a dog, cat, horse, cow, pig, turkey, goat, fish, monkey, chicken, rat, mouse, and sheep.

The invention further provides compositions that comprise an inert TLR ligand and an immune stimulating complex. Various embodiments recited above apply equally to the compositions of the invention and will not be recited again. The components of the composition together may be provided in amounts effective to stimulate an innate immune response. The composition may be pharmaceutically acceptable, and consistent with this, it may further comprise a pharmaceutically acceptable carrier. The composition is preferably further formulated for parenteral administration such as intramuscular administration or subcutaneous administration.

In yet another aspect, the invention provides a method for manufacturing a medicament comprising an inert TLR ligand and an immune stimulating complex, preferably for stimulating an innate immune response.

In another aspect, the invention provides a method for reducing tumor size, comprising administering to a subject in need thereof a CpG oligonucleotide, for example one comprising a nucleotide sequence of 5′ TCGTCGTTTTGTCGTTTTGTCGTT 3′ (SEQ ID NO: 1), and an immune stimulating complex, and an anti-cancer agent, in an amount effective to reduce tumor size. The CpG oligonucleotide and the immune stimulating complex are administered by a route different from the anti-cancer agent. In one embodiment, the ratio of CpG oligonucleotide to immune stimulating complex is 100:1 or 20:1.

In yet another aspect, the invention provides a method for reducing tumor size, comprising administering to a subject in need thereof a CpG oligonucleotide, for example one comprising a nucleotide sequence of 5′ TCGTCGTTTTGTCGTTTTGTCGTT 3′ (SEQ ID NO: 1), and an immune stimulating complex, and an anti-cancer agent, in an amount effective to reduce tumor size, wherein the CpG oligonucleotide and the immune stimulating complex are present in a ratio of 20:1 or 100:1. In one-embodiment, the CpG oligonucleotide and the immune stimulating complex are administered in a route different from the anti-cancer therapy.

Similar embodiments apply to these and various other aspects of the invention. These embodiments are recited below and it is to be understood that they apply equally to different aspects of the invention.

Thus, in one embodiment, the CpG oligonucleotide has a modified phosphate backbone. The modified phosphate backbone may be partially or wholly modified. Alternatively, the modified phosphate backbone may comprise a phosphorothioate modification. The oligonucleotide may comprise a palindrome.

In another embodiment, the subject has a cancer selected from the group consisting of biliary tract cancer, bone cancer, brain and CNS cancer, breast cancer, cervical cancer, choriocarcinoma, colon cancer, connective tissue cancer, endometrial cancer, esophageal cancer, eye cancer, gastric cancer, Hodgkin's lymphoma, intraepithelial neoplasm, larynx cancer, liver cancer, lung cancer such as small cell lung cancer and non-small cell lung cancer, lymphoma, melanoma, neuroblastoma, oral cancer, oral cavity cancer, ovarian cancer, pancreatic cancer, prostate cancer, rectal cancer, renal cancer, skin cancer, testicular cancer and thyroid cancer.

In another embodiment, the CpG oligonucleotide and the immune stimulating complex are administered parenterally, such as subcutaneously. The anti-cancer agent may be administered intra-peritoneally, orally or intravenously.

The CpG oligonucleotide may be mixed together with the immune stimulating complex prior to administration.

In one embodiment, the immune stimulating complex further comprises a phospholipid. In another embodiment, the CpG oligonucleotide is sterol-linked, phospholipid-linked or glycoside-linked. In one embodiment, the glycoside-linked CpG oligonucleotide is a saponin-linked CpG oligonucleotide. In related embodiments, the sterol-linked CpG oligonucleotide replaces a sterol in the immune stimulating complex or the saponin-linked CpG oligonucleotide replaces a saponin in the immune stimulating complex.

In one embodiment, the anti-cancer agent is a chemotherapeutic agent. The chemotherapeutic agent may be taxol, cisplatin, carboplatin, 5-fluorouracil (5-FU), paclitaxel such as oral paclitaxel, oral taxoid, capecitabine, or gemcitabine.

In another embodiment, the anti-cancer therapy is an immunotherapeutic agent. The immunotherapeutic agent may be herceptin, C225, anti-VEGF, MDX-210, MDX-220, or EMD-72000.

In some embodiments, the anti-cancer agent is administered weekly, including on day to day 35. In some embodiments, the CpG oligonucleotide and immune stimulating complex is administered on alternating days, and/or weekly, including on days 1, 3, 7 and thereafter weekly for a period of one, two or more months.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use herein of “including”, “comprising”, “having”, “containing”, “involving”, and variations thereof, is meant to encompass the items listed thereafter and equivalents thereof as well as additional items.

### BRIEF DESCRIPTION OF THE DRAWINGS

The Figures are illustrative only and are not required for enablement of the invention disclosed herein.

FIG. 1A is a graph showing % survival as a function of time for animals receiving CpG 7909 formulated with IMX in a model of renal cell carcinoma.

FIG. 1B is a graph showing tumor volume as a function of time for animals receiving CpG 7909 formulated with IMX in a model of renal cell carcinoma.

FIG. 2A is a graph showing % survival as a function of time for animals receiving CpG 7909 formulated with IMX in a model of NSCLC.

FIG. 2B is a graph showing tumor volume as a function of time for animals receiving CpG 7909 formulated with IMX in a model of NSCLC.

FIG. 3 is a graph showing % survival as a function of time for animals receiving CpG 7909 formulated with IMX combined with Taxol in a model of NSCLC.

FIG. 4 is a graph showing tumor volume as a function of time for animals receiving CpG 7909 formulated with IMX combined with Taxol in a model of NSCLC.

### DETAILED DESCRIPTION OF THE INVENTION

The invention relates broadly to the particular formulations as unexpectedly efficient delivery vehicles for TLR ligands. The formulations comprise immune stimulating complexes the comprise sterols and saponins. Examples of suitable immune stimulating complexes include ISCOM® and ISCOMATRIX® adjuvants both of which are commercially available from CSL Limited (Parkville, Victoria, Australia). The invention is premised in part on the unexpected discovery that immune stimulating complexes are a particularly effective vehicle for delivery of TLR ligands, particularly those that would be immunologically inert or poorly immunostimulatory if not administered together with the immune stimulating complexes. Although not intending to be bound by any particular mechanism, it is postulated that the immune stimulating complexes enhance delivery of such ligands to their respective receptors (e.g., particular TLR family members) and/or to particular cells irrespective of receptor involvement. This has resulted in the observed synergistic enhancement of innate immune responses when the ligand/complex formulation is used in particular experimental therapeutic settings.

It was unexpected that use of immune stimulating complexes could essentially transform previously-characterized immunologically inert TLR ligands (e.g., oligonucleotides) into immunostimulatory ligands (e.g., oligonucleotides). This observation broadens the genus of TLR ligands (e.g., oligonucleotides) that can be used for immunostimulatory purposes to include oligonucleotides with no previously characterized immunostimulatory motif and/or no or low previously characterized immunostimulatory potential. This finding was completely unexpected. It was further unexpected that the synergy observed for the inert TLR ligand and immune stimulating complex combination was much greater than the level of synergy observed for such ligands when combined with other non-nucleic acid adjuvants and delivery systems.

As described in the Examples in greater detail, co-administration of an immune stimulating complex (e.g., ISCOMATRIX® adjuvant) with a CpG immunostimulatory oligonucleotide having the sequence TCGTCGTTTTGTCGTTTTGTCGTT; SEQ ID NO: 1; ODN 7909 resulted in resulted in increased survival and controlled tumor growth better than did either agent alone when tested in a renal cell carcinoma model.

Importantly, these observations were made in murine cancer models, indicating the therapeutic utility of the formulations provided herein for at least cancer therapies.

Thus, the addition of immune stimulating complexes to immunostimulatory or immunologically “inert” oligonucleotides results in the induction of strong innate immune responses, as indicated by the ability of these combinations to impact upon the therapeutic outcome of tumor-bearing subjects. These findings are unexpected at least in part because of the immunologically “inert” character of some of the oligonucleotides tested.

These findings indicate that formulations comprising immune stimulating complexes and oligonucleotides are useful in optimizing innate immune therapies, such as but not limited to those directed to infectious disease, cancers, allergy and asthma.

Immune stimulating complexes are particles having a diameter ranging in size from 10 nm to 100 nm, and more commonly from 30 nm to 50 nm, and comprised of glycosides and sterols which form a matrix onto which antigens (when used) may multimerize. The complexes can function as adjuvants as well as antigen and non-antigen delivery systems. In the methods and Examples described herein, no exogenous antigen was used or indeed needed. The combined use of the TLR ligands and the immune stimulating complex was sufficient for the subject's innate immune system to recognize the experimentally induced cancers as foreign.

The immune stimulating complexes contain glycosides such as Quillaja saponins, sterols (such as cholesterol), and they may optionally also contain phospholipids (such as but not limited to phosphatidylcholine and phosphatidylethanolamine). Preferably, the glycoside is ISCOPREP® saponin which is a purified saponin fraction derivable from Quil A which is obtained from the bark of the Quillaja saponaria tree. Immune stimulating complex formation is described in greater detail in EP 109942 A and EP 231039 A. Immune stimulating complexes can also be prepared as described in U.S. Pat. No. 5,178,860. The entire contents of these references are incorporated herein by reference. In some embodiments, there is no free saponin or free sterol in the formulations.

The invention embraces the use of non-antigen containing complexes (i.e., complexes that are not “loaded” or combined with antigen prior to administration to a subject). The immune stimulating complexes can be prepared at research scale using well known techniques described in the literature (Morein et al., 1989, In: Vaccines: Recent trends and Progress, Gergoriadis et al. (Eds.), Plenum Press, New York, pp. 153-161; Cox et al., 1997, In: Vaccine Design: The Role of Cytokine Networks, Gergoriadis et al, (Eds.), Plenum Press, New York, pp. 33-49; Coulter et al., 1998, Vaccine 16:1243-1253). The complexes can also be prepared at large scale using well known techniques described in the literature (Kersten et al., 2004, In: Novel Vaccination Strategies, Kaufmann (Ed.), WILEY-VCH, Germany).

The TLR ligands can be formulated with the immune stimulating complexes in any number of ways. For example, the TLR ligands can simply be mixed with the immune stimulating complexes. Alternatively, the TLR ligands can themselves be part of the matrix of the complex, for example by contributing one or more of the components of the matrix. As an example, the TLR ligand may be conjugated to a sterol such as cholesterol. The TLR ligand by conjugation to the sterol then can become part of the matrix of the complex. The TLR ligand may alternatively or additionally be conjugated to other substances such as hydrophobic molecules (e.g., palmitic acid, oleic acid, linoleic acid, and the like). Oligonucleotides conjugated in this manner may then be incorporated into the complex with the hydrophobic molecule contributing to the matrix of the complex. Examples and synthesis of oligonucleotide-lipid conjugates are described in greater detail in U.S. Provisional Patent Application 60/505,977 filed Sep. 25, 2003, the entire contents of which are incorporated by reference herein.

With respect to TLR ligand formulations, the ratio of TLR ligand to immune stimulating complex can range from 100:1 to 1:100. In preferred embodiments, the ratio is 1:1, 3:1, 10:1 or 20:1.

One component of the formulations and compositions of the invention is a TLR ligand. As used herein, a TLR ligand is a molecule that binds to a TLR (i.e., a Toll-like receptor). There are a number of TLR identified to date including TLR1, TLR2, TLR3, TLR4, TLR5, TLR6, TLR7, TLR8, TLR9, TLR10 and TLR11. There are similarly a number of TLR ligands identified to date, some of which have been observed to be immunostimulatory (e.g., CpG oligonucleotides). The invention intends to embrace TLR ligands that have been previously identified as being TLR ligands but which have also been observed to be immunologically inert. As used herein, an immunologically inert TLR ligand is one which has been observed to have no or low immunostimulatory potential. The invention also intends to embrace compounds that according to the invention are tested in the presence and absence of an immune stimulating complex and found to be transformed from an inert compound to an immunostimulatory compound. In some embodiments, the TLR ligands are oligonucleotides that do not possess previously characterized immunostimulatory motifs such as but not limited to unmethylated CpG motifs, methylated CpG motifs, poly T motifs, T-rich motifs, poly-G motifs and the like. Examples of immunostimulatory motifs are described in greater detail in U.S. patent application Publication Nos. U.S. 2003/018406 A1 and U.S. 2003/0212026 A1, published Sep. 25, 2003 and Nov. 13, 2003, respectively, the contents of which are incorporated herein by reference in their entirety. However, immunologically inert species of these latter classes of oligonucleotides (i.e., those possessing a previously characterized immunostimulatory motifs) may be rendered immunostimulatory by combining them with immune stimulating complexes, as described herein.

Screening assays for TLR ligands have been described in U.S. patent application Publication No. U.S. 2003/0104523, published Jun. 5, 2003, the entire contents of which are incorporated herein in their entirety. The invention intends to embrace the use of compounds that are shown to be TLR ligands (e.g., via radiolabeled ligand-receptor assays) but which when compared to, for example, immunostimulatory oligonucleotides appear to be inert because their relative immunostimulatory potential is negligible or therapeutically non-useful in comparison.

One category of such inert TLR ligands is those which in the absence of an immune stimulating complex have no or low immunostimulatory potential but which when formulated with an immune stimulating complex demonstrate at least a 2-fold, at least a 3-fold, at least a 4-fold, at least a 5-fold, at least a 10-fold, at least a 20-fold, at least a 50-fold, or more increase in immunostimulatory potential, as measured by assays known in the art.

Some inert TLR ligands would demonstrate an activity in the absence of an immune stimulating complex that is about that of a true negative control (e.g., saline solution or compound demonstrating no complex-induced increase in immunostimulatory potential). They may demonstrate an immunostimulatory potential that is within 5%, within 10%, within 25%, within 50%, or within 75% of a true negative control.

In some important embodiments, the TLR ligands are TLR3 ligands, TLR7 ligands, TLR8 ligands and TLR9 ligands.

It is possible that many agents previously screened and characterized as non-TLR ligands are in fact TLR ligands which simply were not immunostimulatory in particular screening assays (e.g., assays that used readouts of TLR signalling rather than TLR binding). The invention intends to embrace various of these previously disregarded compounds provided that when combined with immune stimulating complexes they readout as immunostimulatory.

The invention intends to embrace oligonucleotides that are DNA or RNA in nature. As a result, the term “oligonucleotides” refers to both oligodeoxynucleotides (DNA) and oligodeoxyribonucleotides (RNA).

Immunostimulatory oligonucleotides as used herein are oligonucleotides that demonstrate immunostimulatory potential even in the absence of an immune stimulating complex. Preferably, these oligonucleotides provide therapeutically effective levels of immunostimulation. Others can be combined with the immune stimulating complexes in order to induce higher and thus therapeutically effective levels of immunostimulation. Examples of immunostimulatory oligonucleotides include CpG immunostimulatory oligonucleotides containing unmethylated as well as methylated CpG dinucleotide motifs, T-rich and poly-T immunostimulatory oligonucleotides, poly-G immunostimulatory oligonucleotides and phosphorothioate immunostimulatory oligonucleotides. Each of these is discussed in greater detail below.

Immunostimulatory oligonucleotides contain specific sequences previously demonstrated to elicit an immune response. These specific sequences are referred to as “immunostimulatory motifs”, and the oligonucleotides that contain at least one immunostimulatory motif are referred to as “immunostimulatory oligonucleotides”. The immunostimulatory motif may be an “internal immunostimulatory motif”. The term “internal immunostimulatory motif” refers to the position of the motif sequence within a longer nucleic acid sequence, which is longer in length than the motif sequence by at least one nucleotide linked to both the 5′ and 3′ ends of the immunostimulatory motif sequence.

Immunostimulatory oligonucleotides when combined with immune stimulating complexes also demonstrate increased immunostimulatory potential, including the ability to increase survival and reduce tumor volume in tumor-bearing subjects. Thus, even those oligonucleotides that are already immunostimulatory benefit from their combination with immune stimulating complexes.

Immunostimulatory oligonucleotides in some instances include CpG immunostimulatory motifs. Such oligonucleotides are referred to as CpG oligonucleotides. A CpG oligonucleotide as used herein refers to an immunostimulatory CpG oligonucleotide, and accordingly these terms are used interchangeably unless otherwise indicated. A CpG immunostimulatory motif can be methylated or unmethylated. Methylation status of the CpG immunostimulatory motif generally refers to the cytosine residue in the dinucleotide. An immunostimulatory oligonucleotide containing at least one unmethylated CpG dinucleotide is a oligonucleotide which contains a 5′ unmethylated cytosine linked by a phosphate bond to a 3′ guanine, and which activates the immune system. An immunostimulatory oligonucleotide containing at least one methylated CpG dinucleotide is a oligonucleotide which contains a 5′ methylated cytosine linked by a phosphate bond to a 3′ guanine, and which activates the immune system. CpG immunostimulatory oligonucleotides may comprise palindromes that in turn may encompass the CpG dinucleotide.

CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

Some immunostimulatory oligonucleotides are free of CpG dinucleotides. Immunostimulatory oligonucleotides which are free of CpG dinucleotides are referred to as non-CpG immunostimulatory oligonucleotides, and they have non-CpG immunostimulatory motifs. Immunostimulatory oligonucleotides can include any combination of methylated and unmethylated CpG and non-CpG immunostimulatory motifs.

Some examples of non-CpG oligonucleotides include

(SEQ ID NO: 19)

T*Z*G*T*Z*G*T*T*T*T*G*T*Z*G*T*T*T*T*G*T*Z*G*T*T;

(SEQ ID NO: 20)

T*G*C*T*G*C*T*T*T*T*G*T*G*C*T*T*T*T*G*T*G*C*T*T;

(SEQ ID NO: 21)

T*G_C*T*G_C*T*T*T*T_G*T*G_C*T*T*T*T*G*T*G_C*T*T;

(SEQ ID NO: 22)

G*T*G*C*T*C*C*T*T*T*G*T*T*G*T*T*C*T*G*T*G*T*T*T;

(SEQ ID NO: 23)

A*A*G*C*A*C*A*A*A*A*G*C*A*C*A*A*A*A*G*C*A*G*C*A;

(SEQ ID NO: 24)

T*G*C*T*G*G*C*C*T*C*C*T*G*G*C*C*T*G*G*T*G*C;

(SEQ ID NO: 25)

T*G*T*G*C*T*T*T*T*T*T*T*T*T*T*T*T*T*T*T;

(SEQ ID NO: 26)

T*C*C*A*G*G*A*C*T*T*C*T*C*T*C*A*G*G*T*T;

(SEQ ID NO: 27)

G*C*C*A*G*G*A*C*A*C*C*T*C*A*C*A*G*G*A*T.

Oligonucleotides that are free of known immunostimulatory motifs, such as those described herein and those known in the art, are referred to herein as non-immunostimulatory motif oligonucleotides. These oligonucleotides lack unmethylated and methylated CpG immunostimulatory motifs, poly-T motifs, poly-G motifs, CpG-like immunostimulatory motifs (as described in U.S. patent application Publication No. U.S. 2003/0181406 A1, published Sep. 25, 2003), and they are also not T-rich (as described in U.S. patent application Publication No. U.S. 2003/0212026, published Nov. 13, 2003).

Different classes of CpG immunostimulatory oligonucleotides have recently been identified. These are referred to as A, B and C class, and are described in greater detail below.

The “A class” CpG immunostimulatory oligonucleotides are characterized functionally by the ability to induce high levels of interferon-alpha and inducing NK cell activation while having minimal effects on B cell activation. Structurally, this class typically has stabilized poly-G sequences at 5′ and 3′ ends. It also has a palindromic phosphodiester CpG dinucleotide-containing sequence of at least 6 nucleotides, but it does not necessarily contain one of the following hexamer palindromes GACGTC, AGCGCT, or AACGTT described by Yamamoto and colleagues. Yamamoto S et al. J. Immunol 148:4072-6 (1992). A class CpG immunostimulatory oligonucleotides and exemplary sequences of this class have been described in U.S. Non-Provisional patent application Ser. No. 09/672,126 and published PCT application PCT/US00/26527 (WO 01/22990), both filed on Sep. 27, 2000.

The “B class” CpG immunostimulatory oligonucleotides are characterized functionally by the ability to activate B cells but is relatively weak in inducing IFN-α and NK cell activation. Structurally, this class typically is fully stabilized and includes an unmethylated CpG dinucleotide, optionally within certain preferred base contexts.

In one embodiment, the invention provides a B class CpG oligonucleotide represented by at least the formula:

5′ X₁X₂CGX₃X₄ 3′

wherein X₁, X₂, X₃, and X₄ are nucleotides. In one embodiment, X₂ is adenine, guanine, or thymine. In another embodiment, X₃ is cytosine, adenine, or thymine.

One category of isolated B class CpG oligonucleotide is represented by at least the formula:

5′ N₁X₁X₂CGX₃X₄N₂ 3′

wherein X₁, X₂, X₃, and X₄ are nucleotides and N is any nucleotide and N₁ and N₂ are nucleic acid sequences composed of from about 0-25 N's each. In one embodiment, X₁X₂ is a dinucleotide selected from the group consisting of GpT, GpG, GpA, ApA, ApT, ApG, CpT, CpA, CpG, TpA, TpT and TpG; and X₃X₄ is a dinucleotide selected from the group consisting of TpT, ApT, TpG, ApG, CpG, TpC, ApC, CpC, TpA, ApA and CpA. Preferably X₁X₂ is GpA or GpT and X₃X₄ is TpT. In other embodiments, X₁ or X₂ or both are purines and X₃ or X₄ or both are pyrimidines or X₁X₂ is GpA and X₃ or X₄ or both are pyrimidines. In one preferred embodiment, X₁X₂ is a dinucleotide selected from the group consisting of TpA, ApA, ApC, ApG and GpG. In yet another embodiment, X₃X₄ is a dinucleotide selected from the group consisting of TpT, TpA, TpG, ApA, ApG, GpA and CpA. X₁X₂, in another embodiment, is a dinucleotide selected from the group consisting of TpT, TpG, ApT, GpC, CpC, CpT, TpC, GpT and CpG; X₃ is a nucleotide selected from the group consisting of A and T, and X₄ is a nucleotide, but when X₁X₂ is TpC, GpT or CpG, X₃X₄ is not TpC, ApT or ApC.

In another preferred embodiment, the CpG oligonucleotide has the sequence 5′ TCN₁TX₁X₂CGX₃X₄ 3′ (SEQ ID NO.: 2). The CpG oligonucleotides, in some embodiments, include X₁X₂ selected from the group consisting of GpT, GpG, GpA and ApA and X₃X₄ selected from the group consisting of TpT, CpT and TpC.

The B class CpG oligonucleotide sequences of the invention are those broadly described above as well as disclosed in published PCT Patent Applications PCT/US95/01570 (WO 96/02555) and PCT/US97/19791 (WO 98/18810), and in U.S. Pat. Nos. 6,194,388, 6,207,646, 6,214,806, 6,218,371, 6,239,116 and 6,339,068. Exemplary sequences include but are not limited to those disclosed in these latter applications and patents.

The “C class” of CpG immunostimulatory oligonucleotides is characterized functionally by the ability to activate B cells and NK cells and induce IFN-α. Structurally, this class typically includes a B class-type immunostimulatory motif sequence, and a GC-rich palindrome or near-palindrome. Some of these oligonucleotides have both a traditional “stimulatory” CpG sequence and a “GC-rich” or “B-cell neutralizing” motif. These combination motif oligonucleotides have immune stimulating effects that fall somewhere between the effects associated with traditional B class CpG oligonucleotides (i.e., strong induction of B cell activation and dendritic cell (DC) activation), and the effects associated with A class CpG ODN (i.e., strong induction of IFN-α and NK cell activation but relatively poor induction of B cell and DC activation). Krieg A M et al. (1995) Nature 374:546-9; Ballas Z K et al. (1996) J. Immunol 157:1840-5; Yamamoto S et al. (1992) J. Immunol 148:4072-6. Moreover, while preferred B class CpG oligonucleotides often have phosphorothioate backbones and preferred A class CpG oligonucleotides have mixed or chimeric backbones, the C class of combination motif immune stimulatory oligonucleotides may have either stabilized, e.g., phosphorothioate, chimeric, or phosphodiester backbones, and in some preferred embodiments, they have semi-soft backbones. This class has been described in U.S. patent application Ser. No. 10/224,523 filed on Aug. 19, 2002, publication No. U.S. 2003/0148976 A1, published Mar. 6, 2003, the entire contents of which are incorporated herein by reference.

One stimulatory domain or motif of the C class CpG oligonucleotide is defined by the formula: 5′ X₁DCGHX₂ 3′. D is a nucleotide other than C. C is cytosine. G is guanine. H is a nucleotide other than G. X₁ and X₂ are any nucleic acid sequence 0 to 10 nucleotides long. X₁ may include a CG, in which case there is preferably a T immediately preceding this CG. In some embodiments, DCG is TCG. X₁ is preferably from 0 to 6 nucleotides in length. In some embodiments, X₂ does not contain any poly G or poly A motifs. In other embodiments, the immunostimulatory oligonucleotide has a poly-T sequence at the 5′ end or at the 3′ end. As used herein, “poly-A” or “poly-T” shall refer to a stretch of four or more consecutive A's or T's respectively, e.g., 5′ AAAA 3′ or 5′ TTTT 3′. As used herein, “poly-G end” shall refer to a stretch of four or more consecutive G's, e.g., 5′ GGGG 3′, occurring at the 5′ end or the 3′ end of a nucleic acid. As used herein, “poly-G oligonucleotide” shall refer to a oligonucleotide having the formula 5′ X₁X₂GGGX₃X₄ 3′ wherein X₁, X₂, X₃, and X₄ are nucleotides and preferably at least one of X₃ and X₄ is a G. Some preferred designs for the B cell stimulatory domain under this formula comprise TTTTTCG, TCG, TTCG, TTTCG, TTTTCG, TCGT, TTCGT, TTTCGT, TCGTCGT.

The second motif of the C class CpG oligonucleotide is referred to as either P or N and is positioned immediately 5′ to X₁ or immediately 3′ to X₂.

N is a B cell neutralizing sequence that begins with a CGG trinucleotide and is at least 10 nucleotides long. A B cell neutralizing motif includes at least one CpG sequence in which the CG is preceded by a C or followed by a G (Krieg A M et al. (1998) Proc Natl Acad Sci USA 95:12631-12636) or is a CG containing DNA sequence in which the C of the CG is methylated. Neutralizing motifs or sequences have some degree of immunostimulatory capability when present in an otherwise non-stimulatory motif, but when present in the context of other immunostimulatory motifs serve to reduce the immunostimulatory potential of the other motifs.

P is a GC-rich palindrome containing sequence at least 10 nucleotides long. As used herein, “palindrome” and equivalently “palindromic sequence” shall refer to an inverted repeat, i.e., a sequence such as ABCDEE′D′C′B′A′ in which A and A′, B and B′, etc., are bases capable of forming the usual Watson-Crick base pairs.

As used herein, “GC-rich palindrome” shall refer to a palindrome having a base composition of at least two-thirds G's and C's. In some embodiments the GC-rich domain is preferably 3′ to the “B cell stimulatory domain”. In the case of a 10-base long GC-rich palindrome, the palindrome thus contains at least 8 G's and C's. In the case of a 12-base long GC-rich palindrome, the palindrome also contains at least 8 G's and C's. In the case of a 14-mer GC-rich palindrome, at least ten bases of the palindrome are G's and C's. In some embodiments the GC-rich palindrome is made up exclusively of G's and C's.

In some embodiments the GC-rich palindrome has a base composition of at least 81% G's and C's. In the case of such a 10-base long GC-rich palindrome, the palindrome thus is made exclusively of G's and C's. In the case of such a 12-base long GC-rich palindrome, it is preferred that at least ten bases (83%) of the palindrome are G's and C's. In some preferred embodiments, a 12-base long GC-rich palindrome is made exclusively of G's and C's. In the case of a 14-mer GC-rich palindrome, at least twelve bases (86%) of the palindrome are G's and C's. In some preferred embodiments, a 14-base long GC-rich palindrome is made exclusively of G's and C's. The C's of a GC-rich palindrome can be unmethylated or they can be methylated.

In general this domain has at least 3 Cs and Gs, more preferably 4 of each, and most preferably 5 or more of each. The number of Cs and Gs in this domain need not be identical.

It is preferred that the Cs and Gs are arranged so that they are able to form a self-complementary duplex, or palindrome, such as CCGCGCGG. This may be interrupted by As or Ts, but it is preferred that the self-complementarity is at least partially preserved as for example in the motifs CGACGTTCGTCG (SEQ ID NO: 3) or CGGCGCCGTGCCG (SEQ ID NO: 4). When complementarity is not preserved, it is preferred that the non-complementary base pairs be TG. In a preferred embodiment there are no more than 3 consecutive bases that are not part of the palindrome, preferably no more than 2, and most preferably only 1. In some embodiments, the GC-rich palindrome includes at least one CGG trimer, at least one CCG trimer, or at least one CGCG tetramer. In other embodiments, the GC-rich palindrome is not CCCCCCGGGGGG (SEQ ID NO: 5) or GGGGGGCCCCCC (SEQ ID NO: 6), CCCCCGGGGG (SEQ ID NO: 7) or GGGGGCCCCC (SEQ ID NO: 8).

At least one of the G's of the GC rich region may be substituted with an inosine (I). In some embodiments, P includes more than one I. 30 The immunostimulatory oligonucleotide may have one of the following formulas 5′ NX₁DCGHX₂ 3′, 5′ X₁DCGHX₂N 3′, 5′ PX₁DCGHX₂ 3′, 5′ X₁DCGHX₂P 3′, 5′ X₁DCGHX₂PX₃ 3′, 5′ X₁DCGHPX₃ 3′, 540 DCGHX₂PX₃ 3′, 5′ TCGHX₂PX₃ 3′, 5′ DCGHPX₃ 3′ or 5′ DCGHP 3′.

Other immunostimulatory oligonucleotides are defined by a formula 5′ N₁PyGN₂P 3′. N₁ is any sequence 1 to 6 nucleotides long. Py is a pyrimidine. G is guanine. N₂ is any sequence 0 to 30 nucleotides long. P is a GC-rich palindrome containing sequence at least TCGHX₁₀ nucleotides long.

N₁ and N₂ may contain more than 50% pyrimidines, and more preferably more than 50% T. N₁ may include a CG, in which case there is preferably a T immediately preceding this CG. In some embodiments, N₁PyG is TCG (such as TCG GCG CGC GCC GTG CTG CTT T, SEQ ID NO: 18), and most preferably a TCGN₂, where N₂ is not G.

N₁PyGN₂P may include one or more inosine (I) nucleotides. Either the C or the G in N1 may be replaced by inosine, but the CpI is preferred to the IpG. For inosine substitutions such as IpG, the optimal activity may be achieved with the use of a “semi-soft” or chimeric backbone, where the linkage between the IG or the CI is phosphodiester. N₁ may include at least one CI, TCI, IG or TIG motif.

In certain embodiments N₁PyCN₂ is a sequence selected from the group consisting of TTTTTCG, TCG, TTCG, TTTCG, TTTTCG, TCGT, TTCGT, TTTCGT, and TCGTCGT.

Some non-limiting examples of C-Class oligonucleotides include:

(SEQ ID NO: 9)

T*C_G*C_G*T*C_G*T*T*C_G*G*C*G*C_G*C*G*C*C*G

(SEQ ID NO: 10)

T*C_G*T*C_G*A*C_G*T*T*C_G*G*C*G*C_G*C*G*C*C*G

(SEQ ID NO: 11)

T*C_G*G*A*C_G*T*T*C_G*G*C*G*C_G*C*G*C*C*G

(SEQ ID NO: 12)

T*C_G*G*A*C_G*T*T*C_G*G*C*G*C*G*C*C*G

(SEQ ID NO: 13)

T*C_G*C_G*T*C_G*T*T*C_G*G*C*G*C*G*C*C*G

(SEQ ID NO: 14)

T*C_G*A*C_G*T*T*C_G*G*C*G*C_G*C*G*C*C*G

(SEQ ID NO: 15)

T*C_G*A*C_G*T*T*C_G*G*C*G*C*G*C*C*G

(SEQ ID NO: 16)

T*C_G*C_G*T*C_G*T*T*C_G*G*C*G*C*C*G

(SEQ ID NO: 17)

T*C_G*C_G*A*C_G*T*T*C_G*G*C*G*C_G*C*G*C*C*G

wherein “*” refers to a phosphorothioate bond and “-” refers to a phosphodiester bond.

Other immunostimulatory oligonucleotides are those that are T-rich and/or which possess poly-T motifs. These are described in greater detail in U.S. patent application Publication U.S. 2003/0212026 A1. Still other immunostimulatory oligonucleotides possess poly-G motifs. These are described in greater detail in published PCT application WO 00/14217, published Mar. 16, 2000. Still other immunostimulatory oligonucleotides are Cp-G-like oligonucleotides and these have been described in greater detail in U.S. patent application Publication U.S. 2003/0181406 A1, published Sep. 25, 2003. The contents of these published applications are incorporated by reference herein in their entirety.

Some aspects of the invention employ non-CpG oligonucleotides that are conjugated to a component of the immune stimulatory complex, such as for example a sterol or a saponin. In these aspects alone, a non-CpG oligonucleotide refers to an oligonucleotide, whether immunostimulatory or not, which lacks an unmethylated CpG motif. Accordingly, T-rich, poly-T, poly-G, methylated CpG and other CpG-like oligonucleotides are embraced by the methods and compositions provided herein, provided they are sterol-linked, glycoside-linked (e.g., saponin-linked), phospholipid-linked, and the like.

The oligonucleotides may be partially resistant to degradation (e.g., are stabilized). A “stabilized oligonucleotide molecule” shall mean an oligonucleotide that is relatively resistant to in vivo degradation (e.g. via an exo- or endo-nuclease). Nucleic acid stabilization can be accomplished via backbone modifications. Oligonucleotides having phosphorothioate linkages provide maximal activity and protect the oligonucleotide from degradation by intracellular exo- and endo-nucleases. Other modified oligonucleotides include phosphodiester modified oligonucleotides, combinations of phosphodiester and phosphorothioate oligonucleotide, methylphosphonate, methylphosphorothioate, phosphorodithioate, p-ethoxy, and combinations thereof.

The oligonucleotides may have a chimeric backbone. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. Since boranophosphonate linkages have been reported to be stabilized relative to phosphodiester linkages, for purposes of the chimeric nature of the backbone, boranophosphonate linkages can be classified either as phosphodiester-like or as stabilized, depending on the context. For example, in one embodiment a chimeric backbone could include at least one phosphodiester (phosphodiester or phosphodiester-like) linkage and at least one boranophosphonate (stabilized) linkage. In another embodiment, a chimeric backbone could include boranophosphonate (phosphodiester or phosphodiester-like) and phosphorothioate (stabilized) linkages. A “stabilized internucleotide linkage” shall mean an internucleotide linkage that is relatively resistant to in vivo degradation (e.g., via an exo- or endo-nuclease), compared to a phosphodiester internucleotide linkage. Preferred stabilized internucleotide linkages include, without limitation, phosphorothioate, phosphorodithioate, methylphosphonate, and methylphosphorothioate. Other stabilized internucleotide linkages include, without limitation, peptide, alkyl, dephospho, and others as described above.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described. Uhlmann E et al. (1990) Chem Rev 90:544; Goodchild J (1990) Bioconjugate Chem 1:165. Methods for preparing chimeric oligonucleotides are also known. For instance patents issued to Uhlmann et al have described such techniques.

Mixed backbone modified ODN may be synthesized using a commercially available DNA synthesizer and standard phosphoramidite chemistry. (F. E. Eckstein, “Oligonucleotides and Analogues—A Practical Approach” IRL Press, Oxford, UK, 1991, and M. D. Matteucci and M. H. Caruthers, Tetrahedron Lett. 21, 719 (1980)) After coupling, PS linkages are introduced by sulfurization using the Beaucage reagent (R. P. Iyer, W. Egan, J. B. Regan and S. L. Beaucage, J. Am. Chem. Soc. 112, 1253 (1990)) (0.075 M in acetonitrile) or phenyl acetyl disulfide (PADS) followed by capping with acetic anhydride, 2,6-lutidine in tetrahydrofurane (1:1:8; v:v:v) and N-methylimidazole (16% in tetrahydrofurane). This capping step is performed after the sulfurization reaction to minimize formation of undesired phosphodiester (PO) linkages at positions where a phosphorothioate linkage should be located. In the case of the introduction of a phosphodiester linkage, e.g. at a CpG dinucleotide, the intermediate phosphorous-III is oxidized by treatment with a solution of iodine in water/pyridine. After cleavage from the solid support and final deprotection by treatment with concentrated ammonia (15 hrs at 50° C.), the ODN are analyzed by HPLC on a Gen-Pak Fax column (Millipore-Waters) using a NaCl-gradient (e.g. buffer A: 10 mM NaH₂PO₄ in acetonitrile/water=1:4/v:v pH 6.8; buffer B: 10 mM NaH₂PO₄, 1.5 M NaCl in acetonitrile/water=1:4/v:v; 5 to 60% B in 30 minutes at 1 ml/min) or by capillary gel electrophoresis. The ODN can be purified by HPLC or by FPLC on a Source High Performance column (Amersham Pharmacia). HPLC-homogeneous fractions are combined and desalted via a C18 column or by ultrafiltration. The ODN was analyzed by MALDI-TOF mass spectrometry to confirm the calculated mass.

The oligonucleotides can also include other modifications. These include nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

In some embodiments, the oligonucleotides may be soft or semi-soft oligonucleotides. A soft oligonucleotide is an oligonucleotide having a partially stabilized backbone, in which phosphodiester or phosphodiester-like internucleotide linkages occur only within and immediately adjacent to at least one internal pyrimidine-purine dinucleotide (YZ). Preferably YZ is YG, a pyrimidine-guanine (YG) dinucleotide. The at least one internal YZ dinucleotide itself has a phosphodiester or phosphodiester-like internucleotide linkage. A phosphodiester or phosphodiester-like internucleotide linkage occurring immediately adjacent to the at least one internal YZ dinucleotide can be 5′, 3′, or both 5′ and 3′ to the at least one internal YZ dinucleotide.

In particular, phosphodiester or phosphodiester-like internucleotide linkages involve “internal dinucleotides”. An internal dinucleotide in general shall mean any pair of adjacent nucleotides connected by an internucleotide linkage, in which neither nucleotide in the pair of nucleotides is a terminal nucleotide, i.e., neither nucleotide in the pair of nucleotides is a nucleotide defining the 5′ or 3′ end of the oligonucleotide. Thus a linear oligonucleotide that is n nucleotides long has a total of n-1 dinucleotides and only n-3 internal dinucleotides. Each internucleotide linkage in an internal dinucleotide is an internal internucleotide linkage. Thus a linear oligonucleotide that is n nucleotides long has a total of n-1 internucleotide linkages and only n-3 internal internucleotide linkages. The strategically placed phosphodiester or phosphodiester-like internucleotide linkages, therefore, refer to phosphodiester or phosphodiester-like internucleotide linkages positioned between any pair of nucleotides in the nucleic acid sequence. In some embodiments the phosphodiester or phosphodiester-like internucleotide linkages are not positioned between either pair of nucleotides closest to the 5′ or 3′ end.

Preferably a phosphodiester or phosphodiester-like internucleotide linkage occurring immediately adjacent to the at least one internal YZ dinucleotide is itself an internal internucleotide linkage. Thus for a sequence N₁ YZ N₂, wherein N₁ and N₂ are each, independent of the other, any single nucleotide, the YZ dinucleotide has a phosphodiester or phosphodiester-like internucleotide linkage, and in addition (a) N₁ and Y are linked by a phosphodiester or phosphodiester-like internucleotide linkage when N₁ is an internal nucleotide, (b) Z and N₂ are linked by a phosphodiester or phosphodiester-like internucleotide linkage when N₂ is an internal nucleotide, or (c) N₁ and Y are linked by a phosphodiester or phosphodiester-like internucleotide linkage when N₁ is an internal nucleotide and Z and N₂ are linked by a phosphodiester or phosphodiester-like internucleotide linkage when N₂ is an internal nucleotide.

Soft oligonucleotides are believed to be relatively susceptible to nuclease cleavage compared to completely stabilized oligonucleotides. Incorporation of at least one nuclease-sensitive internucleotide linkage, particularly near the middle of the oligonucleotide, is believed to provide an “off switch” which alters the pharmacokinetics of the oligonucleotide so as to reduce the duration of maximal immunostimulatory activity of the oligonucleotide. This can be of particular value in tissues and in clinical applications in which it is desirable to avoid injury related to chronic local inflammation or immunostimulation, e.g., the kidney.

Semi-soft oligonucleotides are oligonucleotides having a partially stabilized backbone, in which phosphodiester or phosphodiester-like internucleotide linkages occur only within at least one internal pyrimidine-purine (YZ) dinucleotide. Semi-soft oligonucleotides may possess increased immunostimulatory potency relative to corresponding fully stabilized oligonucleotides. Due to the greater potency of semi-soft oligonucleotides, semi-soft oligonucleotides may be used, in some instances, at lower effective concentations and have lower effective doses than conventional fully stabilized oligonucleotides in order to achieve a desired biological effect.

It is believed that the foregoing properties of semi-soft oligonucleotides generally increase with increasing “dose” of phosphodiester or phosphodiester-like internucleotide linkages involving internal YZ dinucleotides. Thus it is believed, for example, that generally for a given oligonucleotide sequence with five internal YZ dinucleotides, an oligonucleotide with five internal phosphodiester or phosphodiester-like YZ internucleotide linkages is more immunostimulatory than an oligonucleotide with four internal phosphodiester or phosphodiester-like YG internucleotide linkages, which in turn is more immunostimulatory than an oligonucleotide with three internal phosphodiester or phosphodiester-like YZ internucleotide linkages, which in turn is more immunostimulatory than an oligonucleotide with two internal phosphodiester or phosphodiester-like YZ internucleotide linkages, which in turn is more immunostimulatory than an oligonucleotide with one internal phosphodiester or phosphodiester-like YZ internucleotide linkage. Importantly, inclusion of even one internal phosphodiester or phosphodiester-like YZ internucleotide linkage is believed to be advantageous over no internal phosphodiester or phosphodiester-like YZ internucleotide linkage. In addition to the number of phosphodiester or phosphodiester-like internucleotide linkages, the position along the length of the nucleic acid can also affect potency.

The soft and semi-soft oligonucleotides will generally include, in addition to the phosphodiester or phosphodiester-like internucleotide linkages at preferred internal positions, 5′ and 3′ ends that are resistant to degradation. Such degradation-resistant ends can involve any suitable modification that results in an increased resistance against exonuclease digestion over corresponding unmodified ends. For instance, the 5′ and 3′ ends can be stabilized by the inclusion there of at least one phosphate modification of the backbone. In a preferred embodiment, the at least one phosphate modification of the backbone at each end is independently a phosphorothioate, phosphorodithioate, methylphosphonate or methylphosphorothioate internucleotide linkage. In another embodiment, the degradation-resistant end includes one or more nucleotide units connected by peptide or amide linkages at the 3′ end.

A phosphodiester internucleotide linkage is the type of linkage characteristic of nucleic acids found in nature. The phosphodiester internucleotide linkage includes a phosphorus atom flanked by two bridging oxygen atoms and bound also by two additional oxygen atoms, one charged and the other uncharged. Phosphodiester internucleotide linkage is particularly preferred when it is important to reduce the tissue half-life of the oligonucleotide.

A phosphodiester-like internucleotide linkage is a phosphorus-containing bridging group that is chemically and/or diastereomerically similar to phosphodiester. Measures of similarity to phosphodiester include susceptibility to nuclease digestion and ability to activate RNAse H. Thus for example phosphodiester, but not phosphorothioate, oligonucleotides are susceptible to nuclease digestion, while both phosphodiester and phosphorothioate oligonucleotides activate RNAse H. In a preferred embodiment the phosphodiester-like internucleotide linkage is boranophosphate (or equivalently, boranophosphonate) linkage.

U.S. Pat. No. 5,177,198; U.S. Pat. No. 5,859,231; U.S. Pat. No. 6,160,109; U.S. Pat. No. 6,207,819; Sergueev et al., (1998) J Am Chem Soc 120:9417-27. In another preferred embodiment the phosphodiester-like internucleotide linkage is diasteromerically pure Rp phosphorothioate. It is believed that diasteromerically pure Rp phosphorothioate is more susceptible to nuclease digestion and is better at activating RNAse H than mixed or diastereomerically pure Sp phosphorothioate. Stereoisomers of CpG oligonucleotides are the subject of published PCT application PCT/JS99/17100 (WO 00/06588). It is to be noted that for purposes of the instant invention, the term “phosphodiester-like intemucleotide linkage“specifically excludes phosphorodithioate and methylphosphonate internucleotide linkages.

As described above the soft and semi-soft oligonucleotides may have phosphodiester like linkages between C and G. One example of a phosphodiester-like linkage is a phosphorothioate linkage in an Rp conformation. Oligonucleotide p-chirality can have apparently opposite effects on the immune activity of a CpG oligonucleotide, depending upon the time point at which activity is measured. At an early time point of 40 minutes, the Rₚ but not the S_{P} stereoisomer of phosphorothioate CpG oligonucleotide induces JNK phosphorylation in mouse spleen cells. In contrast, when assayed at a late time point of 44 hr, the S_{P} but not the Rₚ stereoisomer is active in stimulating spleen cell proliferation. This difference in the kinetics and bioactivity of the Rₚ and S_{P} stereoisomers does not result from any difference in cell uptake, but rather most likely is due to two opposing biologic roles of the p-chirality. First, the enhanced activity of the Rp stereoisomer compared to the Sp for stimulating immune cells at early time points indicates that the Rp may be more effective at interacting with the CpG receptor, TLR9, or inducing the downstream signaling pathways. On the other hand, the faster degradation of the Rp PS-oligonucleotides compared to the Sp results in a much shorter duration of signaling, so that the Sp PS-oligonucleotides appear to be more biologically active when tested at later time points.

A surprisingly strong effect is achieved by the p-chirality at the CpG dinucleotide itself. In comparison to a stereo-random CpG oligonucleotide, the congener in which the single CpG dinucleotide was linked in Rₚ was slightly more active, while the congener containing an S_{P} linkage was nearly inactive for inducing spleen cell proliferation.

The size of the oligonucleotide (i.e., the number of nucleotide residues along the length of the oligonucleotide) may also contribute to the stimulatory activity of the oligonucleotide. For facilitating uptake into cells, oligonucleotides preferably have a minimum length of 6 nucleotide residues. Oligonucleotides of any size greater than 6 nucleotides (even many kb long) are capable of inducing an immune response, since larger oligonucleotides are degraded inside cells. It is believed that semi-soft oligonucleotides as short as 4 nucleotides can also be immunostimulatory if they can be delivered to the interior of a cell. In certain preferred embodiments, the oligonucleotides are 4 to 100 nucleotides long, 6 to 100 nucleotides long, or 8 to 100 nucleotides long. In typical embodiments the immunostimulatory oligonucleotides are 4 to 40 nucleotides long, 6 to 40 nucleotides long, or 8 to 40 nucleotides long. In important embodiments, nucleic acids and oligonucleotides of the invention are not plasmids nor expression vectors.

The term oligonucleotide also encompasses oligonucleotides with substitutions or modifications, such as in the bases and/or sugars. For example, they include oligonucleotides having backbone sugars that are covalently attached to low molecular weight organic groups other than a hydroxyl group at the 2′ position and other than a phosphate group or hydroxy group at the 5′ position. Thus modified oligonucleotides may include a 2′-O-alkylated ribose group. In addition, modified oligonucleotides may include sugars such as arabinose or 2′-fluoroarabinose instead of ribose. Thus the oligonucleotides may be heterogeneous in backbone composition thereby containing any possible combination of polymer units linked together such as peptide-nucleic acids (which have an amino acid backbone with nucleic acid bases). The foregoing applies equally to nucleic acids disclosed herein.

The oligonucleotides can encompass various chemical modifications and substitutions, in comparison to natural RNA and DNA, involving a phosphodiester intemucleotide bridge, a β-D-ribose unit and/or a natural nucleotide base (adenine, guanine, cytosine, thymine, uracil). Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke S T et al. (1996) Annu Rev Pharmacol Toxicol 36:107-129; and Hunziker J et al. (1995) Mod Synth Methods 7:331-417. An oligonucleotide may have one or more modifications, wherein each modification is located at a particular phosphodiester intemucleotide bridge and/or at a particular β-D-ribose unit and/or at a particular natural nucleotide base position in comparison to an oligonucleotide of the same sequence which is composed of natural DNA or RNA.

For example, the invention relates to an oligonucleotide which may comprise one or more modifications and wherein each modification is independently selected from
a) the replacement of a phosphodiester internucleotide bridge located at the 3′ and/or the 5′ end of a nucleotide by a modified internucleotide bridge,
b) the replacement of phosphodiester bridge located at the 3′ and/or the 5′ end of a nucleotide by a dephospho bridge,
c) the replacement of a sugar phosphate unit from the sugar phosphate backbone by another unit,
d) the replacement of a β-D-ribose unit by a modified sugar unit, and
e) the replacement of a natural nucleotide base by a modified nucleotide base.

More detailed examples for the chemical modification of an oligonucleotide are as follows.

A phosphodiester intemucleotide bridge located at the 3′ and/or the 5′ end of a nucleotide can be replaced by a modified intemucleotide bridge, wherein the modified intemucleotide bridge is for example selected from phosphorothioate, phosphorodithioate, NR¹R²-phosphoramidate, boranophosphate, α-hydroxybenzyl phosphonate, phosphate-(C₁-C₂₁)-O-alkyl ester, phosphate-[(C₆-C₁₂)aryl-(C₁ -C₂₁)-O-alkyl]ester, (C₁ -C₈)alkylphosphonate and/or (C₆-C₁₂)arylphosphonate bridges, (C₇-C₁₂)-α-hydroxymethyl-aryl (e.g., disclosed in WO 95/01363), wherein (C₆-C₁₂)aryl, (C₆-C₂₀)aryl and (C₆-C₁₄)aryl are optionally substituted by halogen, alkyl, alkoxy, nitro, cyano, and where R¹ and R² are, independently of each other, hydrogen, (C₁-C₁₈)-alkyl, (C₆-C₂₀)-aryl, (C₆-C₁₄)-aryl-(C₁-C₈)-alkyl, preferably hydrogen, (C₁-C₈)-alkyl, preferably (C₁-C₄)-alkyl and/or methoxyethyl, or R¹ and R² form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleotide by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

A sugar phosphate unit (i.e., β-D-ribose and phosphodiester internucleotide bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al. (1989) Nucleic Acids Res 17:6129-41), that is, e.g., the replacement by a morpholino-derivative unit; or to build up a polyamide nucleic acid (“PNA”; as described for example, in Nielsen P E et al. (1994) Bioconjug Chem 5:3-7), that is, e.g., the replacement by a PNA backbone unit, e.g., by 2-aminoethylglycine.

A β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O—(C₁-C₆)alkyl-ribose, preferably 2′-O—(C₁-C₆)alkyl-ribose is 2′-O-methylribose, 2′-O—(C₂-C₆)alkenyl-ribose, 2′-[O—(C₁-C₆)alkyl-O—(C₁-C₆)alkyl]-ribose, 2′-NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

In some preferred embodiments, the sugar is 2′-O-methylribose, particularly for one or both nucleotides linked by a phosphodiester or phosphodiester-like intemucleotide linkage.

Oligonucleotides also include substituted purines and pyrimidines such as C-5 propyne pyrimidine and 7-deaza-7-substituted purine modified bases. Wagner R W et al. (1996) Nat Biotechnol 14:840-4. Besides the more common naturally occurring bases of adenine, cytosine, guanine, thymine, and uracil, the oligonucleotides may also comprise other naturally and non-naturally occurring bases, substituted and unsubstituted aromatic moieties. A modified base is any base which is chemically distinct from the naturally occurring bases typically found in DNA and RNA such as thymine, adenine, cytosine, guanine and uracil, but which share basic chemical structures with these naturally occurring bases. Modified nucleotide bases include, for example, 5-(C₂-C₆)-alkenylcytosine, 5-(C₂-C₆)-alkenyluracil, N4-alkylcytosine, e.g., N4-ethylcytosine, N4-alkyldeoxycytidine, e.g., N4-ethyldeoxycytidine, 5-(C₁-C₆)-alkylcytosine, 5-(C₁-C₆)-alkyluracil, 5-(C₂-C₆)-alkynylcytosine, 5-(C₂-C₆)-alkynyluracil, 2-amino-6-chloropurine, 2-aminopurine, 5-aminouracil, 8-azapurine, 5-bromocytosine, 5-bromouracil, 5-chlorocytosine, 5-chlorouracil, deoxyribonucleotides of nitropyrrole, diaminopurine e.g., 2,4-diaminopurine and 2,6-diaminopurine, dihydrouracil, N²-dimethylguanine, 5-fluorocytosine, 5-fluorouracil, 5-hydroxycytosine, 5-hydroxydeoxycytidine, 5-hydroxymethylcytosine, 5-hydroxymethyldeoxycytidine, 5-hydroxymethyluracil, hypoxanthine, inosine, 5-methylcytosine, C5-propynylpyrimidine, pseudouracil, a substituted 7-deazapurine, preferably 7-deaza-7-substituted and/or 7-deaza-8-substituted purine, 6-thiodeoxyguanosine, 2-thiouracil, 4-thiouracil, uracil, etc. This list is meant to be exemplary and is not to be interpreted to be limiting. Other such modifications are known to those of skill in the art.

In particular formulas described herein a set of modified bases is defined. For instance, the letter Y is used to refer to a nucleotide containing a cytosine or a modified cytosine. A modified cytosine as used herein is a naturally occurring or non-naturally occurring pyrimidine base analog of cytosine which can replace this base without impairing the activity of the oligonucleotide. Modified cytosines include but are not limited to 5-substituted cytosines (e.g. 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). Some of the preferred cytosines include 5-methylcytosine, 5-fluorocytosine, 5-hydroxycytosine, 5-hydroxymethyl-cytosine, and N4-ethylcytosine. In another embodiment, the cytosine base is substituted by a universal base (e.g. 3-nitropyrrole, P-base), an aromatic ring system (e.g. fluorobenzene or difluorobenzene) or a hydrogen atom (dSpacer).

The letter Z is used to refer to guanine or a modified guanine base. A modified guanine as used herein is a naturally occurring or non-naturally occurring purine base analog of guanine which can replace this base without impairing the activity of the oligonucleotide. Modified guanines include but are not limited to 7-deazaguanine, 7-deaza-7-substituted guanine (such as 7-deaza-7-(C2-C6)alkynylguanine), 7-deaza-8-substituted guanine, hypoxanthine, N2-substituted guanines (e.g. N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g. N6-methyl-adenine, 8-oxo-adenine) 8-substituted guanine (e.g. 8-hydroxyguanine and 8-bromoguanine), and 6-thioguanine. In another embodiment of the invention, the guanine base is substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom (dSpacer).

The oligonucleotides may have one or more accessible 5′ ends. It is possible to create modified oligonucleotides having two such 5′ ends. This may be achieved, for instance by attaching two oligonucleotides through a 3′-3′ linkage to generate an oligonucleotide having one or two accessible 5′ ends. The 3′3′-linkage may be a phosphodiester, phosphorothioate or any other modified internucleotide bridge. Methods for accomplishing such linkages are known in the art. For instance, such linkages have been described in Seliger, H.; et al., Oligonucleotide analogs with terminal 3′-3′- and 5′-5′-internucleotidic linkages as antisense inhibitors of viral gene expression, Nucleotides & Nucleotides (1991), 10(1-3), 469-77 and Jiang, et al., Pseudo-cyclic oligonucleotides: in vitro and in vivo properties, Bioorganic & Medicinal Chemistry (1999), 7(12), 2727-2735.

Additionally, 3′3′-linked oligonucleotides where the linkage between the 3′-terminal nucleotides is not a phosphodiester, phosphorothioate or other modified bridge, can be prepared using an additional spacer, such as tri- or tetra-ethylenglycol phosphate moiety (Durand, M. et al, Triple-helix formation by an oligonucleotide containing one (dA)12 and two (dT)12 sequences bridged by two hexaethylene glycol chains, Biochemistry (1992), 31(38), 9197-204, U.S. Pat. No. 5,658,738, and U.S. Pat. No. 5668265). Alternatively, the non-nucleotidic linker may be derived from ethanediol, propanediol, or from an abasic deoxyribose (dSpacer) unit (Fontanel, Marie Laurence et al., Sterical recognition by T4 polynucleotide kinase of non-nucleosidic moieties 5′-attached to oligonucleotides; Nucleic Acids Research (1994), 22(11), 2022-7) using standard phosphoramidite chemistry. The non-nucleotidic linkers can be incorporated once or multiple times, or combined with each other allowing for any desirable distance between the 3′-ends of the two ODNs to be linked.

The oligonucleotides may also contain one or more unusual linkages between the nucleotide or nucleotide-analogous moieties. The usual internucleoside linkage is a 3′5′-linkage. All other linkages are considered to be unusual internucleoside linkages, such as 2′5′-, 5′5′-, 3′3′-, 2′2′-, 2′3′-linkages. The nomenclature 2′to 5′ is chosen according to the carbon atom of ribose. However, if unnatural sugar moieties are employed, such as ring-expanded sugar analogs (e.g. hexanose, cyclohexene or pyranose) or bi- or tricyclic sugar analogs, then this nomenclature changes according to the nomenclature of the monomer. In 3′-deoxy-β-D-ribopyranose analogs (also called p-DNA), the mononucleotides are e.g. connected via a 4′2′-linkage.

If the oligonucleotide contains one 3′3′-linkage, then this oligonucleotide may have two unlinked 5′-ends. Similarly, if the oligonucleotide contains one 5′5′-linkage, then this oligonucleotide may have two unlinked 3′-ends. The accessibility of unlinked ends of nucleotides may be better accessible by their receptors. Both types of unusual linkages (3′3′- and 5′5′) were described by Ramalho Ortigao et al. (Antisense Research and Development (1992) 2, 129-46), whereby oligonucleotides having a 3′3′-linkage were reported to show enhanced stability towards cleavage by nucleases.

Different types of linkages can also be combined in one molecule which may lead to branching of the oligomer. If one part of the oligonucleotide is connected at the 3′-end via a 3′3′-linkage to a second oligonucleotide part and at the 2′-end via a 2′3′-linkage to a third part of the molecule, this results e.g. in a branched oligonucleotide with three 5′-ends (3′3′-, 2′3′-branched).

In principle, linkages between different parts of an oligonucleotide or between different oligonucleotides, respectively, can occur via all parts of the molecule, as long as this does not negatively interfere with the recognition by its receptor. According to the nature of the oligonucleotide, the linkage can involve the sugar moiety (Su), the heterocyclic nucleobase (Ba) or the phosphate backbone (Ph). Thus, linkages of the type Su-Su, Su-Ph, Su-Ba, Ba-Ba, Ba-Su, Ba-Ph, Ph-Ph, Ph-Su, and Ph-Ba are possible. If the oligonucleotides are further modified by certain non-nucleotidic substituents, the linkage can also occur via the modified parts of the oligonucleotides. These modifications also include modified oligonucleotides, e.g. PNA, LNA, or Morpholino Oligonucleotide analogs.

The linkages are preferably composed of C, H, N, O, S, B, P, and Halogen, containing 3 to 300 atoms. An example with 3 atoms is an acetyl linkage (ODN1-3′-O—CH₂—O-3′-ODN2) connecting e.g. the 3′-hydroxy group of one nucleotide to the 3′-hydroxy group of a second oligonucleotide. An example with about 300 atoms is PEG-40 (tetraconta polyethyleneglycol). Preferred linkages are phosphodiester, phosphorothioate, methylphosphonate, phosphoramidate, boranophosphonate, amide, ether, thioether, acetal, thioacetal, urea, thiourea, sulfonamide, Schiff' Base and disulfide linkages. It is also possible to use the Solulink BioConjugation System available at the “trilinkbiotech” website.

If the oligonucleotide is composed of two or more sequence parts, these parts can be identical or different. Thus, in an oligonucleotide with a 3′3′-linkage, the sequences can be identical 5′-ODN1-3′3′-ODN1-5′ or different 5′-ODN1-3′3′-ODN2-5′. Furthermore, the chemical modification of the various oligonucleotide parts as well as the linker connecting them may be different. Since the uptake of short oligonucleotides appears to be less efficient than that of long oligonucleotides, linking of two or more short sequences results in improved immune stimulation. The length of the short oligonucleotides is preferably 2-20 nucleotides, more preferably 3-16 nucleotides, but most preferably 5-10 nucleotides. Preferred are linked oligonucleotides which have two or more unlinked 5′-ends.

The oligonucleotide partial sequences may also be linked by non-nucleotidic linkers, in particular abasic linkers (dSpacers), trietyhlene glycol units or hexaethylene glycol units. Further preferred linkers are alkylamino linkers, such as C3, C6, C12 aminolinkers, and also alkylthiol linkers, such as C3 or C6 thiol linkers. The oligonucleotides can also be linked by aromatic residues which may be further substituted by alkyl or substituted alkyl groups. The oligonucleotides may also contain a Doubler or Trebler unit as described at the “glenres” website, in particular those oligonucleotides with a 3′3′-linkage. Branching of the oligonucleotides by multiple doubler, trebler, or other multiplier units leads to dendrimers which are a further embodiment of this invention. The oligonucleotides may also contain linker units resulting from peptide modifying reagents or oligonucleotide modifying reagents as described at the “glenres” website. Furthermore, it may contain one or more natural or unnatural amino acid residues which are connected by peptide (amide) linkages.

Another possibility for linking oligonucleotides is via crosslinking of the heterocyclic bases (Verma and Eckstein; Annu. Rev. Biochem. (1998) 67: 99-134; page 124). A linkage between the sugar moiety of one sequence part with the heterocyclic base of another sequence part (Iyer et al. Curr. Opin. Mol. Therapeutics (1999) 1: 344-358; page 352) may also be used.

The different oligonucleotides are synthesized by established methods and can be linked together on-line during solid-phase synthesis. Alternatively, they may be linked together post-synthesis of the individual partial sequences.

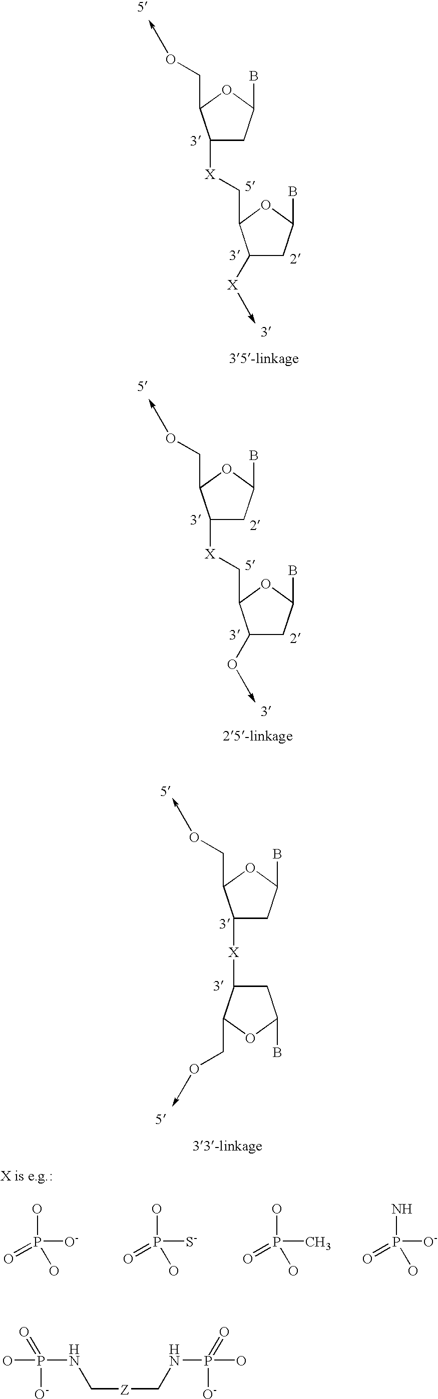

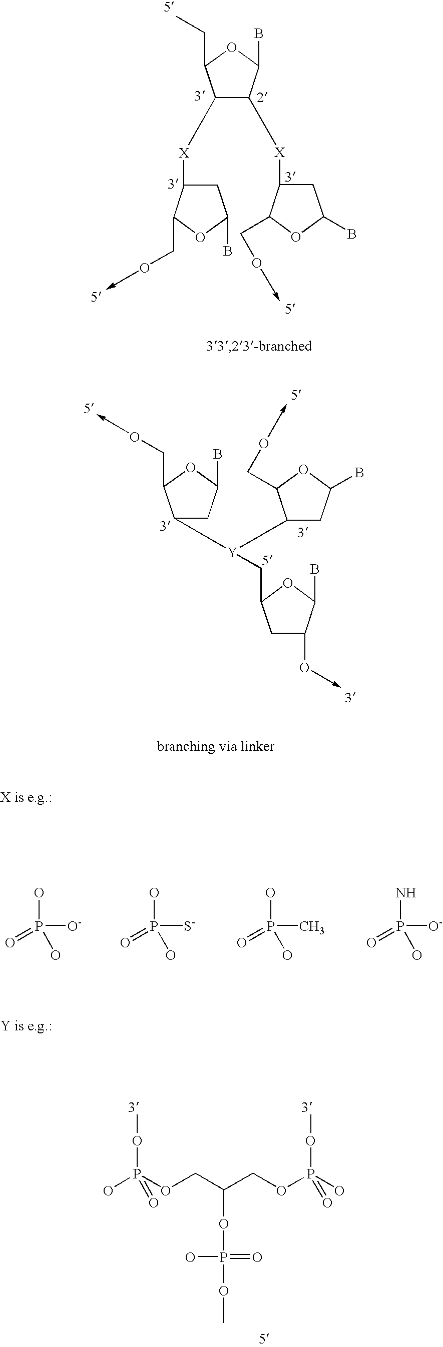

An isolated form is one in which the substance has been physically separated from the components with which it is normally exists or can be found.

The term substantially purified, as used herein, refers to a substance which is substantially free of proteins, lipids, carbohydrates or other materials with which it is naturally associated. One skilled in the art can purify viral or bacterial polypeptides using standard techniques for protein purification. A substantially pure polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the viral or bacterial polypeptide can also be determined by amino-terminal amino acid sequence analysis.

The TLR ligands are also commonly used in their isolated forms. An isolated oligonucleotide is an oligonucleotide that is physically separated from those substances with which it is normally associated. If the oligonucleotide is produced from naturally occurring sources, then it is isolated if it is physically separated from other components of that naturally occurring source such as cells, proteins, nuclei, chromosomes, etc.

As used herein, the terms treat, treated, or treating when used with respect to an disorder, such as an infectious disease, cancer or allergy, refers to prophylactic treatment which increases the resistance of a subject to development of the disease (e.g., to infection with a pathogen) or, in other words, decreases the likelihood that the subject will develop the disease (e.g., become infected with the pathogen) as well as a therapeutic treatment after the subject has developed the disease in order to fight the disease (e.g., reduce or eliminate the infection) or prevent the disease from becoming worse.

The formulations described herein are useful therapeutically and prophylactically for stimulating the immune system to form innate immune responses necessary to treat cancer, infectious disease, allergy, asthma and other disorders. The formulations demonstrate unexpectedly better immune stimulatory effects as compared to other adjuvant combinations.

A subject shall mean a human or vertebrate animal including but not limited to a dog, cat, horse, cow, pig, sheep, goat, turkey, chicken, primate, e.g., monkey, and fish (aquaculture species), e.g. salmon. The invention can be used to treat cancer and tumors, infections, and allergy/asthma in human and non-human subjects. Cancer is one of the leading causes of death in companion animals (e.g., cats and dogs).

Because innate immunity developed in part to protect a host against foreign antigens, such as for example, foreign pathogens, the methods of the invention are suited in some instances to treating subjects that are at risk of contacting foreign pathogens. In such subjects, the subject may be administered the TLR ligand and the immune stimulating complex on a regular basis when that risk is greatest, i.e., during allergy season or after exposure to a cancer causing agent. Additionally the TLR ligand and immune stimulating complex may be administered to travelers before they travel to foreign lands where they are at risk of exposure to infectious agents. Likewise the TLR ligand and immune stimulating complex may be administered to soldiers or civilians at risk of exposure to biowarfare.

A subject at risk, as used herein, is a subject who has a higher than normal risk of developing an infection, or a cancer, or an allergy.

A subject at risk of developing an infection may be a subject who is planning to travel to an area where a particular type of infectious agent is prevalent or it may be a subject who through lifestyle or medical procedures is exposed to bodily fluids which may contain infectious organisms or directly to the organism or even any subject living in an area where an infectious organism has been identified. Subjects at risk of developing infection also include general populations to which a medical agency recommends vaccination with a particular microbial antigen.

A subject having an infection is a subject that has been exposed to an infectious pathogen and has acute or chronic detectable levels of the pathogen in the body. An infectious disease, as used herein, is a disease arising from the presence of a foreign microorganism in the body. It is particularly important to develop effective innate immunity strategies and treatments to protect the body's mucosal surfaces, which are the primary site of pathogenic entry.

The infectious disease may be a bacterial infection, a viral infection, a fungal infection, a parasitic infection, or a mycobacterial infection, although it is not so limited. Examples of these are listed herein and supplemented below.

The bacterial infection may be but is not limited to an Actinomyces infection, an anthrax infection, a Bacteriodes infection, a Borrelia infection, a Campylobacter infection, a Citrobacter infection, a Clostridium difficile infection, a Corynebacterium infection, an E. coli infection, an Enterobacter infection, a Gardnerella infection, a Haemophilus infection, an H. pylori infection, a Klebsiella infection, a Legionella infection, a Listeria infection, a Neisseria infection, a Nocardia infection, a Pasteurella infection, a Pneumococcus infection, a Proteus infection, a Pseudomonas infection, a Salmonella infection, a Shigella infection, a Spirillum infection, a Spirochaeta infection, a Staphylococcal infection, a Streptobacillus infection, a Streptococcal infection, and a Treponema infection.

Gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

The viral infection may be but is not limited to an adenovirus infection, a retrovirus infection, a rotavirus infection, etc. It may be but is not limited to a cytomegalovirus infection, an Epstein Barr virus infection, a hepatitis A virus infection, a hepatitis B virus infection, a hepatitis C virus infection, a Herpes simplex virus 1 infection, a Herpes simplex virus 2 infection, an HIV infection, a human papilloma virus infection, an influenza A virus infection, a monkey pox infection, a respiratory syncytial virus infection, a SARS infection a small pox infection, a varicella-zoster virus infection. In some embodiments, the infectious disease is a chronic infectious disease such as a chronic viral infection. Examples include hepatitis virus infection, human papilloma virus infection, HIV infection, and Herpes simplex virus infection.

Categories of viruses that have been found in humans include but are not limited to Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

The fungal infection may be but is not limited to aspergillosis, blastomycosis, candidiasis, chromomycosis, crytococcosis, histoplasmosis, mycetoma infections, paracoccidioidomycosis, pseudallescheriasis, ringworm, and tinea versicolor infection.

Examples of fungi include Cryptococcus neoformans, Histoplasma capsulatum, Coccidioides immitis, Blastomyces dermatitidis, Chlamydia trachomatis, and Candida albicans.

The mycobacterial infection may be but is not limited to M. tuberculosis and M. leprae.

The parasitic infection may be but is not limited to amebiasis, Echinococcus infections, Fascioliasis, Hymenolepsis infection, Leishmaniasis, Onchocerciasis, Necator americanus infection, neurocysticercosis, Paragonimiasis, Plasmodium infections, Pneumocystis infection, Schistosomiasis, Taenia infection, Trichomonas vaginalis infection, Trichuris trichuria infection, Trypanosoma brucei infection and Trypanosoma cruzi infection.

Parasites include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference.

A subject at risk of developing allergy or asthma includes those subjects that have been identified as having an allergy or asthma but that don't have the active disease during the oligonucleotide treatment as well as subjects that are considered to be at risk of developing these diseases because of genetic or environmental factors. If the subject may be exposed to an allergen, e.g., during pollen season, then that subject is at risk of exposure to the allergen.

A subject having an allergy is a subject that has or is at risk of developing an allergic reaction in response to an allergen. An allergy refers to acquired hypersensitivity to a substance (allergen). Allergic conditions include but are not limited to eczema, allergic rhinitis or coryza, hay fever, conjunctivitis, bronchial asthma, urticaria (hives) and food allergies, and other atopic conditions.

A subject at risk of developing a cancer is one who has a higher than normal probability of developing cancer (i.e., higher than the probability in the general population). These subjects include, for instance, subjects having a genetic abnormality, the presence of which has been demonstrated to have a correlative relation to a higher than normal likelihood of developing a cancer and subjects exposed to cancer causing agents such as tobacco, asbestos, or other chemical toxins, or a subject who has previously been treated for cancer that is in apparent remission.

A subject having a cancer is a subject that has detectable cancerous cells. Immunostimulatory oligonucleotides, particularly unmethylated CpG immunostimulatory oligonucleotides have been shown to induce innate immunity in tumor-bearing subjects when administered together with immune stimulating complexes. The induced innate immunity has been sufficient to reduce tumor volume and increase survival rates in such subjects. Subcutaneous administration of the oligonucleotide and complex appeared better at inducing such a response as compared to intraperitoneal administration. Further combination with at least one anti-cancer agent appeared to render better outcomes in reduction in tumor volumes and increased survival, but not to statistically significant levels.

The cancer may be a carcinoma or a sarcoma but it is not so limited. For example, the cancer may be basal cell carcinoma, biliary tract cancer, bladder cancer, bone cancer, brain cancer, breast cancer, cervical cancer, choriocarcinoma, CNS cancer, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, acute lymphoid leukemia, acute myeloid leukemia, chronic lymphoid leukemia, chronic myeloid leukemia, cutaneous T-cell leukemia, hairy cell leukemia, liver cancer, non-small cell lung cancer, small cell lung cancer, lymphoma, follicular lymphoma, Hodgkin's lymphoma, Non-Hodgkin's lymphoma, melanoma, myeloma, multiple myeloma, neuroblastoma, oral cavity cancer, ovarian cancer, pancreatic cancer, prostate cancer, rectal cancer, renal cancer, cancer of the respiratory system, retinoblastoma, rhabdomyosarcoma, skin cancer, squamous cell carcinoma, stomach cancer, testicular cancer, thyroid cancer, cancer of the urinary system and uterine cancer.

The invention can also be used to treat cancer and tumors in non human subjects. Cancer is one of the leading causes of death in companion animals (i.e., cats and dogs). Cancer usually strikes older animals which, in the case of house pets, have become integrated into the family. Forty-five % of dogs older than 10 years of age, are likely to succumb to the disease. The most common treatment options include surgery, chemotherapy and radiation therapy. Others treatment modalities which have been used with some success are laser therapy, cryotherapy, hyperthermia and immunotherapy. The choice of treatment depends on type of cancer and degree of dissemination. Unless the malignant growth is confined to a discrete area in the body, it is difficult to remove only malignant tissue without also affecting normal cells.

Malignant disorders commonly diagnosed in dogs and cats include but are not limited to lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma. Other neoplasias in dogs include genital squamous cell carcinoma, transmissable veneral tumor, testicular tumor, seminoma, Sertoli cell tumor, hemangiopericytoma, histiocytoma, chloroma (granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma. Additional malignancies diagnosed in cats include follicular lymphoma, intestinal lymphosarcoma, fibrosarcoma and pulmonary squamous cell carcinoma. The ferret, an ever-more popular house pet is known to develop insulinoma, lymphoma, sarcoma, neuroma, pancreatic islet cell tumor, gastric MALT lymphoma and gastric adenocarcinoma.

Neoplasias affecting agricultural livestock include leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulendotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lumphadenitis (CLA): chronic, infectious, contagious disease of sheep and goats caused by the bacterium Corynebacterium pseudotuberculosis, and contagious lung tumor of sheep caused by jaagsiekte.

Prion diseases include a number of fatal, neurodegenerative diseases believed to be caused by aggregates of normal protein that is present in an abnormal conformation. The normal prion protein is usually present in the cell membrane of many tissues, particularly neuronal tissue. The abnormally conformed prion protein is believed to be directly involved in converting normally conformed prion protein into more of the abnormally conformed prion protein, which then self-assembles into aggregates that are damaging to neuronal tissue anatomy and function.

At least some of the prion diseases are transmissible. However, unlike bacteria, viruses, fungi, parasites, and other replicating pathogens, transmissible prions are simply proteins; they are transmissible without any accompanying nucleic acid. For reasons that are not yet fully understood, the abnormally conformed prion proteins generally do not induce an immune response. Thus, exposure of a healthy individual to abnormally conformed prion protein can initiate a prion disease that can go unchecked by the immune system.

The formulations of the invention are useful in the treatment of prion diseases, including Creutzfeldt-Jakob disease (CJD), bovine spongiform encephalopathy (BSE), and scrapie. The CJD may be iatrogenic CJD (iCJD), variant CJD (vCJD) or sporadic CJD (sCJD). The formulations are also useful in the treatment of other neurologic diseases involving abnormal protein deposits or aggregates. Such diseases include Alzheimer's disease, which involves deposits of amyloid. The main component of amyloid plaques is amyloid-beta peptide (Abeta), a fibrillar 40-42 amino acid peptide that accumulates extracellularly and causes neuronal death. Further reference to prion diseases, subjects at risk thereof and diagnosis of subjects having prior disease can be found in published PCT Application WO 2004/007743, published Jan. 22, 2004, the entire contents of which are recited herein in their entirety.

The subjects may be further administered other therapeutic agents or regimens. Examples include anti-microbial agents, anti-cancer agents, anti-allergy agents and anti-asthma agents. These other agents may be formulated together with or separately from the TLR ligand/complex formulations of the invention.

An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. Anti-microbial agents include but are not limited to anti-bacterial agents, anti-viral agents, anti-fungal agents, anti-parasitic agents, and anti-mycobacterial agents. Phrases such as “anti-infective agent”, “anti-bacterial agent”, “anti-viral agent”, “anti-fungal agent”, “anti-parasitic agent”, “parasiticide” and anti-mycobacterial agent” have established meanings to those of ordinary skill in the art and are defined in standard medical texts.

Anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites. Anti-mycobacterial agents kill or inhibit mycobacteria.

Anti-bacterial agents kill or inhibit the growth or function of bacteria. A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics. Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors.

Anti-viral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleotide analogues), maturation of new virus proteins (e.g. protease inhibitors), and budding and release of the virus.

Anti-virals that are nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, zidovudine (azidothymidine), imiquimod, and resimiquimod.

Anti-viral agents useful in the invention include but are not limited to immunoglobulins, amantadine, interferons, nucleotide analogues, and protease inhibitors. Specific examples of anti-virals include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

Anti-parasitic agents, also referred to as parasiticides, useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

The formulations may also be administered in conjunction with an anti-cancer agent. An anti-cancer agent is an agent that is administered to a subject for the purpose of treating a cancer, and preferably is cytotoxic, particularly to proliferating cells. For the purpose of this specification, anti-cancer agents are classified as chemotherapeutic agents, immunotherapeutic agents, hormone therapy, and biological response modifiers.

The chemotherapeutic agent may be selected from the group consisting of methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS famesyl transferase inhibitor, famesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, ISI641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Paclitaxel, Taxol/Paclitaxel, Xeloda/Capecitabine, Furtulon/Doxifluridine, Cyclopax/oral paclitaxel, Oral Taxoid,-SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levaminsole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate, but it is not so limited.

Antibodies directed to cancer antigens include but are not limited to Rituxan™, Herceptin™, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART ID10 Ab, SMART ABL 364 Ab and ImmuRAIT-CEA.

Anti-asthma/allergy agents may be selected from the group consisting of PDE-4 inhibitor, Bronchodilator/beta-2 agonist, K+ channel opener, VLA-4 antagonist, Neurokin antagonist, TXA2 synthesis inhibitor, Xanthanine, Arachidonic acid antagonist, 5 lipoxygenase inhibitor, Thromboxin A2 receptor antagonist, Thromboxane A2 antagonist, Inhibitor of 5-lipox activation protein, and Protease inhibitor, but is not so limited. In some important embodiments, the asthma/allergy medicament is a Bronchodilator/beta-2 agonist selected from the group consisting of salmeterol, salbutamol, terbutaline, D2522/formoterol, fenoterol, and orciprenaline.

The anti-asthma/allergy agent may also be Anti-histamines and Prostaglandin inducers. In one embodiment, the anti-histamine is selected from the group consisting of loratidine, cetirizine, buclizine, ceterizine analogues, fexofenadine, terfenadine, desloratadine, norastemizole, epinastine, ebastine, ebastine, astemizole, levocabastine, azelastine, tranilast, terfenadine, mizolastine, betatastine, CS 560, and HSR 609. In another embodiment, the Prostaglandin inducer is S-5751.

The anti-asthma/allergy agents may also be Steroids and Immunomodulators. The immunomodulators may be selected from the group consisting of anti-inflammatory agents, leukotriene antagonists, IL-4 muteins, Soluble IL-4 receptors, Immunosuppressants, anti-IL-4 antibodies, IL-4 antagonists, anti-IL-5 antibodies, soluble IL-1 3 receptor-Fc fusion proteins, anti-IL-9 antibodies, CCR3 antagonists, CCR5 antagonists, VLA-4 inhibitors, and Downregulators of IgE, but are not so limited. In one embodiment, the downregulator of IgE is an anti-IgE. The steroid may be beclomethasone, fluticasone, tramcinolone, budesonide, and budesonide.

Cytokines or B-7 co-stimulatory molecules (Bueler & Mulligan, 1996; Chow et al., 1997; Geissler et al., 1997; Iwasaki et al., 1997; Kim et al., 1997; Iwasaki et al., 1997; Tsuji et al., 1997) may also be administered to the subjects being treated, either together with or separate from the TLR ligand/complex formulations. The term cytokine is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (G-CSF), IFN-α, tumor necrosis factor (TNF), TGF-β, FLT-3 ligand, and CD40 ligand.

The agents of the invention may be administered simultaneously or sequentially with the other therapeutic agents and/or regimens. When the other therapeutic agents are administered substantially simultaneously with the agents of the invention, they can be administered in the same or separate formulations, provided they are administered at substantially the same time (i.e., generally within minutes of each other, or within the time it takes a person of ordinary skill in the medical or pharmaceutical arts to administer the two substances). When the other therapeutic agents are administered sequentially with the agents of the invention, then the administration of the other therapeutic agents and the agents is temporally separated. The separation in time between the administration of these compounds may be a matter of minutes, hours, days or longer.

The term effective amount of a composition or of its constituents refers to the amount necessary or sufficient to realize a desired biologic effect. For example, an effective amount of an TLR ligand/complex formulation for inducing an innate immune response is that amount necessary to activate NK cell activity, stimulate production and/or secretion of one of the innate immunity cytokines disclosed herein, or ultimately to evidence some clinical change (e.g., a reduction in tumor volume or an increase in survival of a tumor-bearing subject).

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the type of disease or condition being treated, the particular oligonucleotide being administered, the dose of immune stimulating complex administered, the size of the subject, or the severity of the disease or condition. One of ordinary skill in the art can empirically determine the effective amount of a particular TLR ligand/complex formulation and/or other therapeutic agent without necessitating undue experimentation.

Subject doses of the compounds described herein typically range from about 0.1 μg to 10 mg per administration, which depending on the application could be given for example daily, weekly, or any other amount of time therebetween. More typically doses range from about 1 μg to 10 mg per administration, even more typically from about 10 μg to 5 mg per administration, still more typically from about 10 μg to 1 mg, and most typically from about 100 μg to 1 mg, with 2-4 administrations being spaced days or weeks apart.

In some aspects of the invention, sub-optimal levels of either or both agents can be used. As used herein, a sub-optimal level of an agent is an amount that if used alone (or at least without the synergizing partner of the invention) would not yield maximal therapeutic benefit, but when used in combination with the synergizing partner would yield maximal therapeutic benefit. The ability to use sub-optimal doses of therapeutic agents is useful because it allows for a reduction in any potential side effects of the therapeutic agents.

A sub-optimal level of a second therapeutic agent may only be needed when it is used together with the TLR ligand/complex formulation of the invention. This is again useful for a number of reasons, including but not limited to reducing the side effects associated with the second therapeutic. As used herein, the second therapeutic refers to the anti-microbial, anti-cancer, anti-asthma/allergy agents (and the like) described herein.

For any compound described herein the therapeutically effective amount can be initially determined from animal models. A therapeutically effective dose can also be determined from human data for immunostimulatory oligonucleotides and complexes which have been tested individually in humans (human clinical trials have been initiated). The applied dose can be adjusted based on the relative bioavailability and potency of the administered compound. Adjusting the dose to achieve maximal efficacy based on the methods described above and other methods are known in the art and within the capabilities of the ordinarily skilled artisan.

The compositions of the invention may be administered neat or in pharmaceutically acceptable solutions, which may in turn contain pharmaceutically acceptable concentrations of salt, buffering agents, preservatives, compatible carriers, and optionally other therapeutic ingredients.

The TLR ligand/complex formulation can be administered to a subject by any mode of administration. Preferred routes of administration include but are not limited to parenteral administrations such as intramuscular and subcutaneous. In some embodiments, the TLR ligand/complex formulation may also be administered via mucosal routes such as oral, nasal, inhalation, rectal, vaginal, and the like.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above components. The components may be chemically modified so that oral delivery of the derivative is efficacious. Generally, the chemical modification contemplated is the attachment of at least one moiety to the component molecule itself, where said moiety permits (a) inhibition of proteolysis; and/or (b) uptake into the blood stream from the stomach or intestine. Also desired is the increase in overall stability of the components and increase in circulation time in the body. Examples of such moieties include polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the oligonucleotide or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the formulations may be contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation as a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the formulations may take the form of tablets or lozenges formulated in conventional manner.

For administration by inhalation, the formulations may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g gelatin for use in an inhaler or insufflator may be formulated containing a powder mix of the compound and a suitable powder base such as lactose or starch.

The formulation may be delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., Mar., (recombinant human growth hormone); Debs et al., 1988, J. Immunol. 140:3482-3488 (IFN-gamma and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The formulations and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric-acid and a salt (0.8-2% w/v). Suitable preservatives include benzalkonium chloride (0.003-0.03% w/v); chlorobutanol (0.3-0.9% w/v); parabens (0.01-0.25% w/v and thimerosal (0.004-0.02% w/v).

The formulations optionally include a pharmaceutically-acceptable carrier. The term pharmaceutically-acceptable carrier means one or more compatible solid or liquid filler, diluents or encapsulating substances which are suitable for administration to a human or other. vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

The following examples demonstrate the therapeutic utility of the combined use of immune stimulating complexes and TLR ligands for inducing innate immunity in experimental murine cancer models.

The anti-tumor effects of immunostimulatory CpG 7909 (TCG TCG TTT TGT CGT TTT GTC GTT; SEQ ID NO: 1) has been demonstrated previously using several murine cancer models. Furthermore, CpG 7909 has been shown to augment the anti-tumor effects of some chemotherapeutic drugs.

Immune stimulating complexes function as adjuvants (particularly in vaccine settings), as well as delivery vehicles and possibly depot effectors. The depot function of immune stimulating complexes appears to play a role in the use of TLR ligands in monotherapy treatment (i.e., non-vaccine treatments).

Materials Generally:

Mice: All experiments were carried out using female BALB/c mice aged 6-8 weeks with 10 mice per experimental or control group.
Oligonucleotides: All oligonucleotides were obtained from Coley Pharmaceutical GmbH, Langenfeld, Germany.
Immune stimulating complexes: ISCOMATRIX® adjuvant, herein referred to as IMX, was the immune stimulating complex used in these Examples. The IMX was prepared at laboratory scale using dialysis, essentially by the method of Morein et al, 1998. Briefly, to 800 μl of phosphate buffered saline (PBS) pH6.2 was added 100 μl of a solution containing 17 mg/ml cholesterol and 10 mg/ml dipalmitoylphosphatidylcholine (DPPC) in 20% w/v Mega-10 then 100 μl of 32 mg/ml ISCOPREP® saponin (CSL Limited, Parkville, Victoria, Australia) in PBS pH6.2 . The solution was held at 25° C. for 1 hour with gentle mixing and then dialysed extensively against PBS pH6.2. During dialysis IMX containing ISCOPREP® saponin, cholesterol and DPPC was formed.
   Experiment 1:

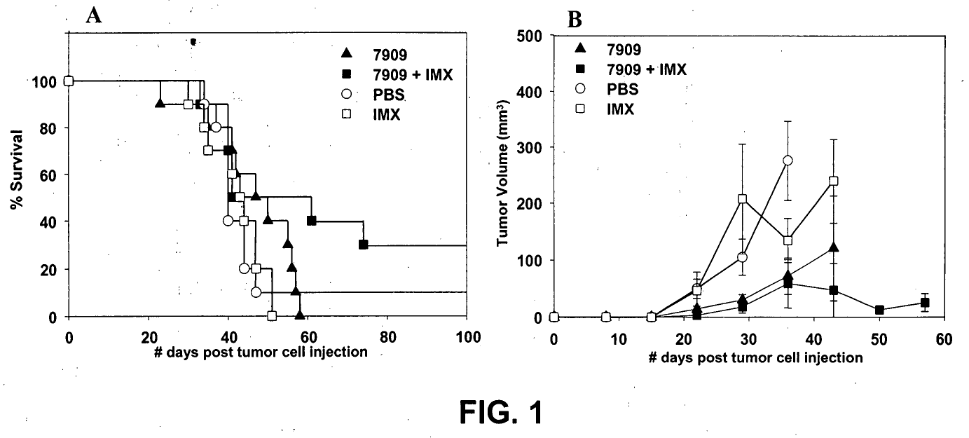

Female BALB/c mice (n=10 per group) were injected with 2×10⁵ Renca (renal carcinoma) cells by SC injection into the left flank. Animals were treated with CpG 7909 alone, ISCOMATRIX® (IMX) alone or a combination of CpG 7909 and INIX administered by SC injection into the tumor perimeter weekly from day 10-28 post tumor cell injection. Control animals were injected with 100 μl of phosphate buffered saline (PBS) weekly from day 10-38 post tumor cell injection. Animals were monitored for survival (FIG. 1, panel A) and tumor growth (FIG. 1, panel B). Tumor size (length and width) was measured using a digital vernier calliper. Tumor volume was calculated by using the formula: Tumor volume=(0.4) (ab2), where a=large diameter and b=smaller diameter. In the tumor volume graph (FIG. 1, panel B), changes in average tumor volume are indicated until 50% death in each animal group.

Experiment 2:

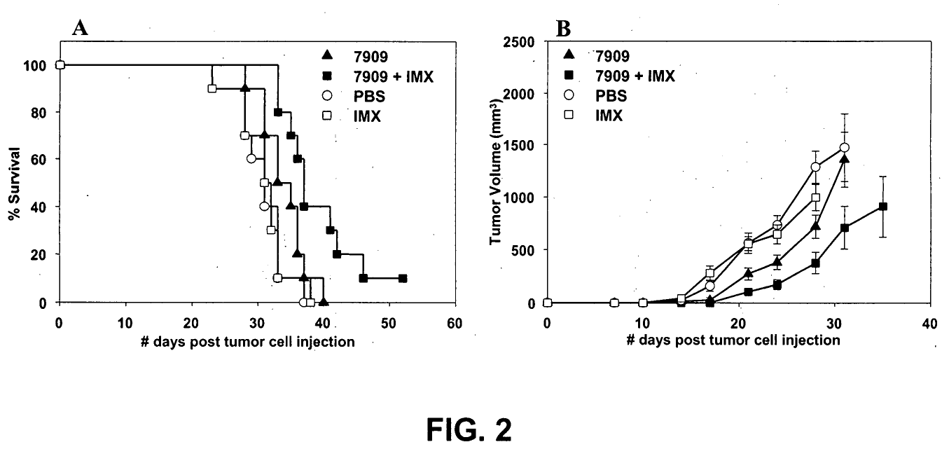

Female C57B1/6 mice (n=10 per group) were injected with 2×10⁶ Lewis lung carcinoma cells by SC injection on day 0. Animals were treated with CpG 7909 alone, IMX alone or a combination of CpG 7909 and IMX administered by SC injection into the tumor perimeter on day 1, 3, 7 and then weekly for 2 months. Animals were monitored for survival (FIG. 2, panel A) and tumor growth (FIG. 2, panel B). Tumor size (length and width) was measured using a digital vernier caliper. Tumor volume was calculated by using the formula: Tumor volume=(0.4) (ab2), where a=large diameter and b=smaller diameter. In the tumor volume graphs (FIG. 2, panel B), changes in average tumor volume are indicated until 50% death in each animal group.

Experiment 3:

The materials, animal groups and treatment schedules are described in the following tables.

Materials:

| Reagent | Source, Lot No | Stock Conc | | Final Conc | | LSP-CDN |
|---|---|---|---|---|---|---|
| Lewis Lung | ATCC# CRL-1642 | N/A | | 2 × 10⁷ | cell/ml | GL015 |
| Carcinoma Cells | | | | | | |
| CPG 7909 | ACZ-01D-006-M | 20.64 | mg/ml | 1 | mg/ml | GL006 |
| Taxol | Bristol-Myers | 6 | mg/ml | 0.7308 | mg/ml | CA001, |
| | Squibb, 3A65544 | | | | | CA002 |
| CSL ISCOMs | 525524F37 | 1.064 | mg/ml | 0.05 | mg/ml | N/A |
| (IMX) | | | | | | |

| | | | | |
|---|---|---|---|---|
| PBS | Sigma, #P0261 | N/A | N/A | N/A |

Animal Groups [C57B1/6]:

| | | | | | IMX | | |
|---|---|---|---|---|---|---|---|
| Grp | Size | Treatment | Route | ODN Dose | Dose | Treatment Day | LLC Cells |
| 2016 | 10 | PBS | SC | 100 μl | | Day 1, 3, 7 & wkly for 2 mths | 2 × 10⁶ |
| 2017 | 10 | 7909 in saline | SC | 100 μg | | Day 1, 3, 7 & wkly for 2 mths | 2 × 10⁶ |
| 2018 | 10 | 7909 in IMX | SC | 100 μg | 5 μg | Day 1, 3, 7 & wkly for 2 mths | 2 × 10⁶ |
| 2019 | 10 | IMX only | SC | | 5 μg | Day 1, 3, 7 & wkly for 2 mths | 2 × 10⁶ |
| 2020 | 10 | Taxol | IP | 36 mg/kg | | Wkly from D7 to D35 | 2 × 10⁶ |
| 2021 | 10 | Taxol | IP | 36 mg/kg | | Wkly from D7 to D35 | 2 × 10⁶ |
| | | 7909 in saline | SC | 100 μg | | Day 1, 3, 7 & wkly for 2 mths | |
| 2022 | 10 | Taxol | IP | 36 mg/kg | 5 μg | Wkly from D7 to D35 | 2 × 10⁶ |
| | | 7909 in IMX | SC | 100 μg | | Day 1, 3, 7 & wkly for 2 mths | |
| 2023 | 10 | (Taxol + 7909) | IP | 36 mg/kg + | 5 μg | Wkly from D7 to Day 35 | 2 × 10⁶ |
| | | in IMX | (all together) | 100 μg | | (combined) | |
| | | 7909 in IMX | SC | 100 μg | | Day 1, 3, 42, 49, 56 | |

Treatment Schedules:

| Day | Group number | Treatment(s) |
|---|---|---|
| 0 | 2016-2023 | Cells injected (SC) |
| 1 | 2016-19/2021-2023 | CPG, placebo (SC) |
| 3 | 2016-19/2021-2023 | CPG, placebo (SC) |
| 7 | 2016-19/2021-2022 | CPG, placebo (SC) |
| | 2020-2023 | Taxol (IP) |
| 14 | 2016-19/2021-2022 | CPG, placebo (SC) |
| | 2020-2023 | Taxol (IP) |
| 21 | 2016-19/2021-2022 | CPG, placebo (SC) |
| | 2020-2023 | Taxol (IP) |
| 28 | 2016-19/2021-2022 | CPG, placebo (SC) |
| | 2020-2023 | Taxol (IP) |
| 35 | 2016-19/2021-2022 | CPG, placebo (SC) |
| | 2020-2023 | Taxol (IP) |
| 42 | 2016-19/2021-2023 | CPG, placebo (SC) |
| 49 | 2016-19/2021-2023 | CPG, placebo (SC) |
| 56 | 2016-19/2021-2023 | CPG, placebo (SC) |

Measurement Schedule: LLC is a fast growing tumor cell line. Measurements will be done twice weekly on Tuesdays and Fridays starting on Day 7.

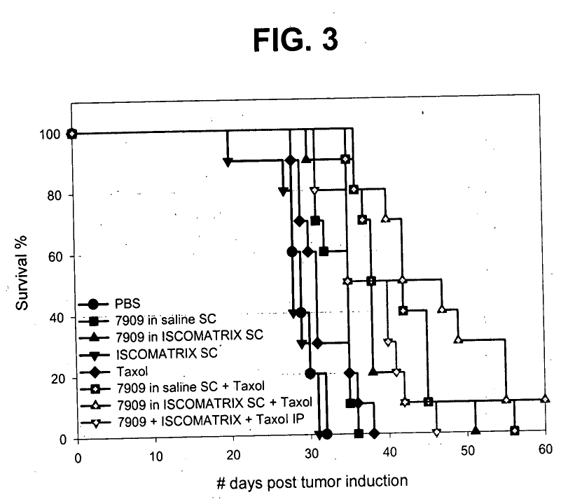

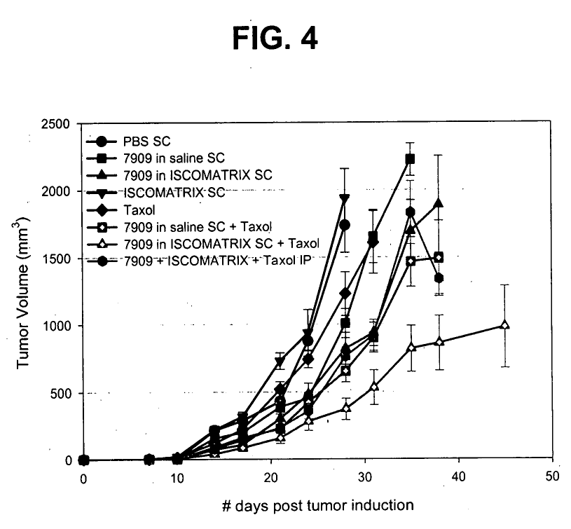

The results are shown in FIG. 3 and FIG. 4.

FIG. 3 corresponds to experiments in which female C57B1/6 mice (n=10 per group) were injected with 2×10⁶ Lewis lung carcinoma cells SC on day 0. Animals were treated with 5 μg IMX, 100 μg CPG 7909 ±5 μg IMX by SC injection alone or in combination with 36 mg/kg Taxol. CpG 7909 was injected on days 1, 3, 7 & weekly for 2 months. Taxol was given IP weekly from day 7 to 35. Combined 7909+IMX+Taxol given IP weekly from day 7 to 35, otherwise 7909+IMX given SC on days 1, 3, 42, 49, 52. The placebo control animals received PBS (100 μl) by SC injection. Animals were monitored for survival.

With respect to survival of tumor-bearing subjects, CpG 7909 and Taxol had similar effects when each was used as a monotherapy (p=0.5). The combination of CpG 7909 and Taxol was more effective than either agent alone, indicating synergy between these agents. And although FIG. 2 shows that CpG 7909, IMX and Taxol did slightly better than CpG 7909 and Taxol together, the difference (at least for these experiments) was not statistically significant. These results indicate at a minimum that immune stimulating complexes do not interfere with synergistic effects seen with the combination of CpG 7909 and Taxol.

With respect to tumor growth as shown in FIG. 4, CpG 7909 and Taxol had similar effects when each was used as a monotherapy (p=0.27). CpG 7909 and Taxol together were more effective than either agent alone. Moreover, CpG 7909, Taxol and IMX controlled tumor growth better than did CpG 7909 when used together with Taxol. Thus, with respect to tumor growth, immune stimulating complexes enhanced the synergy between CpG 7909 and Taxol.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method for inducing an innate immune response comprising
administering to a subject, in need thereof, an inert TLR ligand and an immune stimulating complex, in an amount effective to induce an innate immune response.

2. The method of claim 1, wherein the innate immune response comprises natural killer (NK) cell activation.

3. The method of claim 1, wherein the inert TLR ligand is an oligonucleotide.

4. The method of claim 3, wherein the oligonucleotide is a ribonucleotide.

5. The method of claim 3, wherein the oligonucleotide is a deoxyribonucleotide.

6. The method of claim 4, wherein the oligonucleotide has a modified phosphate backbone.

7. The method of claim 6, wherein the modified phosphate backbone is partially or wholly modified.

8. The method of claim 6, wherein the modified phosphate backbone comprises a phosphorothioate modification.

9. The method of claim 3, wherein the oligonucleotide comprises a palindrome.

10. The method of claim 1, wherein the subject has or is at risk of developing a cancer.

11. (canceled)

12. The method of claim 1, wherein the subject has or is at risk of developing an infection.

13-16. (canceled)

17. The method of claim 1, wherein the subject has or is at risk of developing an allergy.

18. The method of claim 1, wherein the inert TLR ligand the immune stimulating complex are administered intramuscularly or subcutaneously.

19. The method of claim 1, wherein the inert TLR ligand is mixed together with the immune stimulating complex prior to administration.

20. The method of claim 1, wherein the immune stimulating complex further comprises a phospholipid.

21. The method of claim 1, wherein the inert TLR ligand is sterol-linked,

22. The method of claim 21, wherein the sterol-linked TLR ligand replaces a sterol in the immune stimulating complex.

23-30. (canceled)

31. A composition comprising
an inert TLR ligand and an immune stimulating complex.

32-52. (canceled)

53. A method for reducing tumor size, comprising
administering to a subject in need thereof a CpG oligonucleotide comprising a nucleotide sequence of

| | |
|---|---|
| 5′ TCGTCGTTTTGTCGTTTTGTCGTT 3′ | (SEQ ID NO: 1) |

and an immune stimulating complex, and an anti-cancer agent in an amount effective to reduce tumor size, wherein the CpG oligonucleotide and the immune stimulating complex are administered by a route different from the anti-cancer agent.

54. (canceled)

55. A method for reducing tumor size, comprising
administering to a subject in need thereof a CpG oligonucleotide comprising a nucleotide sequence of

| | |
|---|---|
| 5′ TCGTCGTTTTGTCGTTTTGTCGTT 3′ | (SEQ ID NO: 1) |

and an immune stimulating complex, and an anti-cancer agent in an amount effective to reduce tumor size, wherein the CpG oligonucleotide and the immune stimulating complex are present in a ratio of 20:1 or 100:1.

56-90. (canceled)
